(19) United States
(12) Patent Application Publication
(10) Pub. No.: US 2018/0028490 A1
(43) Pub. Date: Feb. 1, 2018

(54) Methods Of Treating Diarrhea And Promoting Intestinal Health In Non-Human Animals

(71) Applicant: JAGUAR HEALTH, INC., San Francisco, CA (US)

(72) Inventors: Serge MARTINOD, Groton, CT (US); Michael Hauser, San Francisco, CA (US)

(21) Appl. No.: 15/551,675

(22) PCT Filed: Feb. 24, 2016
(86) PCT No.: PCT/US16/19348

§ 371 Date: Aug. 17, 2017

Related U.S. Application Data
(60) Provisional application No. 62/121,308, filed on Feb. 26, 2015.
(60) Provisional application No. 62/172,573, filed on Jun. 8, 2015.
(60) Provisional application No. 62/245,862, filed on Oct. 23, 2015.
(60) Provisional application No. 62/255,146, filed on Nov. 13, 2015.

(51) Int. Cl. A61K 31/353; A61K 9/08; A23K 50/10; A61K 9/06; A61K 35/741; A23K 20/121; A61K 36/47; A61K 9/00

(52) U.S. Cl. CPC A61K 31/353; A61K 36/47; A61K 9/08; A61K 9/0053; A61K 9/0095; A61K 9/06; A61K 9/0068; A61K 35/741; A23K 20/121; A23K 50/10; A61K 2035/11

(57) ABSTRACT

Methods of treating neonatal and young non-human animals suffering from diarrhea, and/or symptoms thereof, by administering to an animal in need thereof a proanthocyanidin polymer composition isolated from a Croton spp. or a Calophyllum spp. are provided. In particular, the neonatal and young animals include calves, young equines and young camels, which frequently suffer from diarrhea of various etiologies, and the administered proanthocyanidin polymer composition is isolated from Croton lechleri. Further provided are methods of improving weight gain and/or reducing mortality in neonatal non-human animals by administration of the proanthocyanidin polymer composition. The composition, either enteric or non-enteric, can be in aqueous soluble form and orally administered to the affected neonatal and young animals. The invention also provides prebiotic compositions of botanical extracts of Croton spp. or of Calophyllum spp. useful to promote a beneficial intestinal microbiota.

### FIELD OF THE INVENTION

The invention relates to the treatment of diarrhea in neonatal, unweaned and young non-human animals with a composition comprising a proanthocyanidin polymer isolated from the plant Croton spp. or Calophyllum spp., or with a latex, extract, or food supplement derived therefrom. More particularly, the composition is effective in treating secretory diarrhea of various etiologies and reducing the severity and duration of diarrhea in neonatal and young, non-human animals. Botanical extracts of Croton spp. or Calophyllum spp. containing polyphenols are also effective to promote a beneficial intestinal microbiota and may be used to promote intestinal health and prevent diarrheal disease in non-human animals. The proanthocyanidin polymer compositions can be administered to neonatal non-human animals to improve weight gain and survivability.

### BACKGROUND OF THE INVENTION

Infectious diseases are among the most widespread problems of neonatal and young animals, such as calves of different animal species. Diarrhea, called “scours” in calves, frequently occurs within the first several days of life and is an important cause of calf sickness and death in the United States. Dehydration from diarrhea in neonatal and young farm or larger-sized animals results in measurable morbidity and mortality in many millions of animals worldwide.

A wide array of infectious and pathogenic agents including bacteria, viruses, and parasites cause diarrhea in animals, particularly, domesticated livestock animals associated with farming, food, and labor. Many of these enteropathogens cause one or more adverse effects in the animals, such as severe intestinal lesions, dehydration, alterations in enzyme activity, and/or alterations in nutrient transport mechanisms. The clinical presentation of diarrhea caused by these agents may vary; some diarrheas are self-limiting, while others are associated with high morbidity or high mortality (R. E. Holland, 1990, Clin. Microbiol. Rev., 3(4):345-375).

Infectious diarrhea of neonatal animals is an extremely common and economically devastating condition confronted by the animal agriculture and animal husbandry industries. When encountered in a herd, acute infectious diarrhea is often difficult to manage, contain and cure, because of the large numbers of potential enteropathogens involved, the differences in natural immunity among animals within the herd, environmental conditions and stresses, nutritional factors, the dynamics of the animal population, management conditions, and a difficulty in determining an etiological diagnosis. As a consequence, such a diagnosis is frequently not established for a large percentage of cases of neonatal animal diarrheas. In addition, neonatal and young animals, such as calves, may be predisposed to diarrheas as a result of difficult births, exposure, poor maternal nutrition and/or health, poor mothering capabilities on the part of the dam, or a combination of these factors. When some or all of these conditions occur, the resistance of the calf to infectious diseases, for example, is lowered, and exposure to and invasion by infectious agents play pivotal roles in producing diarrhea.

Some of the most common types of infectious agents resulting in neonatal animal diarrhea, particularly in calves, include E. coli, e.g., E. coli K-99; rotavirus and coronavirus, cryptosporidia, Salmonella spp., Campylobacter jejuni. In some cases, such as infection by Salmonella, which is a human pathogen, human handlers of the animals and those who treat the animals, may also be at risk of infection and disease.

Diarrhea in neonatal and young animals can also be due to noninfectious causes, such as changes to a feeding program, energy deficiencies and vitamin shortages related to pregnant adult females that can extend to the newborn offspring, causing weakness and susceptibility to infection. Environmental and sanitation conditions associated with the birth of newborn animals can also be associated with outbreaks of disease and resulting diarrhea. For example, an unclean environment, e.g., an accumulation of urine and manure in an area where animals are born and nursed, can lead to disease syndromes that are characterized by diarrhea. In addition, problems related to giving birth by adult females, such as difficult calving and insufficient colostrum, can lead to weak newborns and a lack of passive immunity provided by the colostrum. Thus, adverse conditions affecting both the mothers and their newly and recently born offspring can lead to outbreaks of diarrhea requiring treatment of the neonatal and young animals. Noninfectious diarrhea, while oftentimes not severe enough to cause death, can weaken the young animal and make it more susceptible to infectious diarrhea, which contributes to a neonatal and young animal's inability to survive.

The available and commonly used treatments for diarrhea in neonatal and young non-human animals typically involve vital fluid replacement and electrolyte replenishment to counter or stop fluid and electrolyte loss. Other types of treatments include the administration of gut-lining protectants, e.g., bismuth, oral antibiotics, and agents that affect gut motility. Depending on the cause(s), timing, severity and course of diarrhea and/or its associated disease or condition, the various known treatments may or may not be effective, and the animals may or may not respond adequately. Because the economic and humane impacts of diarrhea and its related conditions on the afflicted animals, their handlers and caregivers are so great, there is a compelling need for alternative, safe, and medically effective, as well as cost effective, treatments and remedies.

Disruptions in the balance of microorganisms in the gut of non-human animals can cause intestinal distress, can inhibit weight gain or maintenance and can lead to intestinal disease and infection. Such disruptions may have numerous causes including poor husbandry conditions, disease, or stressors, such as transport of the non-human animal. Promoting and establishing an appropriate balance of beneficial microorganisms in the intestinal tract can improve the health and vitality of non-human animals and can prevent or ameliorate intestinal disease. Agents with a prebiotic effect can be administered to establish and promote such a balance of beneficial microorganisms. There is a need in the art for such agents for veterinary use.

### SUMMARY OF THE INVENTION

The present invention relates to methods of treating diarrhea in neonatal, young, or non-adult animals in need thereof by administering a polymeric proanthocyanidin, i.e., a proanthocyanidin polymer, from a Croton species or Calophyllum species. In an embodiment, a pharmaceutically or physiologically acceptable formulation or composition comprising a proanthocyanidin polymer from a Croton species or Calophyllum species is administered. In particular embodiments, a proanthocyanidin polymer from Croton lechleri, or pharmaceutically acceptable formulation or composition comprising a proanthocyanidin polymer from Croton lechleri is administered. In addition, polyphenol-containing botanical extracts of Croton and Calophyllum species, including the proanthocyanidin polymer compositions, have prebiotic activity. Accordingly, the invention provides prebiotic compositions of botanical extracts of Croton and Calophyllum species, particularly botanical extracts of C. lechleri, that contain polyphenols. In an embodiment, treatment of non-human subjects, e.g., pre-weaned calves, having diarrhea with a C. lechleri-derived proanthocyanidin product, such as botanical extracts of C. lechleri or SB-300, supports a beneficial prebiotic mechanism of the product on the optimization of the intestinal microbiome profile in treated subjects. In an embodiment, treatment with enteric-coated SB-300 results in a higher relative abundance of the Faecalibacterium and Bifidobacterium bacterial genera, which comprise a number of species of probiotic bacteria, in animals after treatment cessation compared with non-treated animals.

In an embodiment, the proanthocyanidin polymer composition is a latex or extract from a Croton species or Calophyllum species, in particular, Croton lechleri. In another embodiment, the composition is a botanical extract of Croton lechleri containing a proanthocyanidin oligomer, or a food supplement formulation of the botanical extract of Croton lechleri. Such Croton species or Calophyllum species latex or extract compositions can be more highly purified as described herein. In an embodiment, the methods involve the administration of a pharmaceutically acceptable composition comprising a proanthocyanidin polymer from Croton lechleri to a non-human animal in need thereof. In an embodiment, the methods involve the administration of a proanthocyanidin polymer from Croton lechleri, or a pharmaceutically acceptable composition comprising a proanthocyanidin polymer from Croton lechleri, wherein the proanthocyanidin polymer or oligomer from C. lechleri is also known as crofelemer (a purified proanthocyanidin oligomer), SP 303, or SB-300, as further described herein. In certain embodiments, the C. lechleri proanthocyanidin polymer, or composition thereof, is in an enteric coated form that protects the proanthocyanidin polymer from the stomach environment of the non-human animal. In other embodiments, the C. lechleri proanthocyanidin polymer, or composition thereof, is in a non-enteric coated form.

The invention provides a method of treating and preventing the debilitating effects of diarrhea in neonatal and young non-human animals. In particular, the methods treat and prevent dehydration associated with water, fluid and electrolyte losses in animals afflicted with diarrhea. The methods of the invention further prevent or reduce the incidence of intestinal lesions, weakness and death in the neonatal and young non-human animals. In other embodiments, the methods treat and prevent diarrhea associated with colitis, including acute colitis, in afflicted animals. Thus, in an embodiment, the methods of the invention provide antisecretory treatments for diarrhea, particularly, secretory or watery diarrhea, in neonatal and young non-human animals.

The invention is more particularly directed to a method of improving gut health and controlling diarrhea in neonatal bovine or camel calves in need thereof by administering a proanthocyanidin polymer from Croton lechleri in an effective amount to control or treat the diarrhea in these animals. In an embodiment the proanthocyanidin polymer is a formulation, composition, or extract from Croton lechleri. In an embodiment, the proanthocyanidin polymer from Croton lechleri is a more highly purified composition containing proanthocyanidin polymer or oligomer, such as crofelemer or SB-300 compositions described herein.

The invention is also more particularly directed to a method of improving gut health, controlling diarrhea and normalizing stool formation in neonatal or young horses (foals) in need thereof by administering a proanthocyanidin polymer from Croton lechleri in an effective amount to control or treat the diarrhea in these animals. In an embodiment the proanthocyanidin polymer is a formulation, composition, or botanical extract from Croton lechleri. In an embodiment, the formulation, composition, or botanical extract from Croton lechleri is in the form of a paste or gel. In a particular embodiment, the paste formulation comprises beads (nano or microparticles) comprising enterically coated SB-300 or SP 303 and is orally administered to foals in need. In an embodiment, the paste formulation comprises beads (nano or microparticles) comprising enterically coated SB-300. In a particular embodiment, the paste comprising SB-300 enteric beads is orally administered to a foal twice daily for three days. In another particular embodiment, the paste comprising SB-300 enteric beads is orally administered to a foal three times daily for three days. In another particular embodiment, the paste comprising SB-300 enteric beads is orally administered to a foal four times daily for three days. In some embodiments, the paste is orally administered two or more times daily for two or more consecutive days. In some embodiments, the paste is orally administered for three or more consecutive days. In an embodiment, the paste comprising SB-300 enteric beads is orally administered to a foal in need at a dose of 2-10 mg/kg twice daily, or three times daily, or four times daily for three days. In an embodiment, the paste comprising SB-300 enteric beads is orally administered to a foal in need at a dose of 2-4 mg/kg twice daily, or three times daily, or four times daily, preferably for three days. In embodiments, the paste comprising SB-300 enteric beads is orally administered to a foal in need at a dose of 2 mg/kg twice daily for three days, or three times daily for three days, or four times daily for three days.

In an aspect, the invention provides a method of treating a neonatal or young non-human animal having diarrhea associated with enteropathogenic infection, the method comprising orally administering to an animal in need thereof a pharmaceutically acceptable composition comprising an aqueous soluble proanthocyanidin polymer from Croton lechleri, wherein the composition is formulated as a bolus or as a reconstituted powder and administered to the animal in an amount of at least 40 mg to 300 mg for consecutive days greater than one day, thereby treating the diarrhea in the neonatal or young animal. In various embodiments of the method, the neonatal or young animal is selected from a bovine calf, a camel calf, a buffalo calf, a bison calf, a lamb, a kid, a foal, or a piglet. In particular embodiments, the neonatal or young animal is a bovine calf or a camel calf. In other embodiments, the neonatal or young animal is an equine foal. In an embodiment, the proanthocyanidin polymer composition is administered twice daily for three consecutive days. In certain embodiments, the diarrhea is secretory or watery diarrhea associated with enteropathogen infection of the animal with one or more of E. coli, rotavirus, or coronavirus. In some embodiments, the diarrhea is episodic. In some embodiments, the animal is additionally infected with Salmonella spp. and/or Cryptosporidia. In embodiments of the method, the proanthocyanidin polymer composition or botanical extract derived from C. lechleri is administered as a powder reconstituted with oral electrolytes, milk or a milk substitute, physiological saline, or water; or as a bolus; or as a paste or gel; or in animal feed. The treated animals, such as calves or foals, can be less than two weeks of age, or two to four weeks of age. In embodiments of the method, the composition is administered to the animal in an amount of at least 30 mg to 350 mg, or in amount of 40 mg, 50 mg, or 250 mg. In other embodiments, the neonatal or young animal is approximately 30 to 50 kg in weight; is a lamb, a kid of approximately 2 to 8 kg in weight, a bovine calf of approximately 30 to 40 kg in weight, or a camel calf of approximately 40 to 50 kg in weight. In a particular embodiment, the proanthocyanidin polymer composition or botanical extract derived from C. lechleri is administered in a paste formulation at a dose of 2 mg/kg, where the approximate body weight of a foal under one year of age is 60 pounds (lb.). In embodiments, the proanthocyanidin polymer is administered as an enteric coated pharmaceutical composition or as a non-enteric coated pharmaceutical composition. In addition, the proanthocyanidin polymer can be SB-300, SP 303, crofelemer and pharmaceutically acceptable compositions thereof.

In another of its aspects, the invention provides a method of treating a neonatal or unweaned equine animal for diarrhea associated with enteropathogenic infection, the method comprising orally administering to the animal a pharmaceutically acceptable composition comprising an aqueous soluble proanthocyanidin polymer from Croton lechleri, wherein the composition is provided in a form selected from a bolus, a reconstituted powder, or a gel or paste, and is administered to the animal in an amount of at least 100 mg for consecutive days greater than one day, thereby treating the diarrhea in the neonatal or unweaned equine animal. In an embodiment, the animal is infected with bacteria, viruses and protozoa, in which the infection induced the diarrhea. In various embodiments, the proanthocyanidin polymer composition is administered to the animal in an amount of at least 250 mg and optionally can be in the form of a gel contained in a delivery device, which can be a syringe. In an embodiment, the gel or paste comprises polymeric microparticles or nanoparticles containing the composition, and the polymeric microparticles or nanoparticles are optionally pH-sensitive. In embodiments, the animal is less than two weeks of age and/or is approximately 30 to 50 kg in weight. In embodiments of the method, the proanthocyanidin polymer is administered as an enteric coated or as a non-enteric coated pharmaceutical composition. In addition, the proanthocyanidin polymer can be SB-300, SP 303, crofelemer and pharmaceutically acceptable compositions thereof. In a particular embodiment, the proanthocyanidin polymer is enterically protected beads, including enteric beads including SB-300 or SP 303.

In another aspect, the methods of the present invention provide prophylactic or preventative treatment of neonatal and young animals against the debilitating effects of diarrheal disease and its associated symptoms, e.g., dehydration and weight loss. In accordance with the invention, a C. lechleri proanthocyanidin polymer composition can be administered to neonatal and young animals at a suitable time after birth to protect the animals from, or reduce the incidence or severity of, diarrhea outbreaks typically caused by infections and environmental conditions. Administering a C. lechleri proanthocyanidin polymer composition to neonatal and young animals can also serve to ameliorate or reduce the risk of the animals' suffering from a more serious or severe form of disease relative to animals that are not provided with the C. lechleri proanthocyanidin polymer composition. In certain embodiments, administration of the C. lechleri proanthocyanidin polymer composition to neonatal animals within 1, 2, 3 or 4 days after birth for a period of 1, 2, 3, 4 or more days can increase weight gain and/or improve survivability in a population of animals, including in bovines, camels, buffalo, bisons, lambs, goats, horses and pigs. The C. lechleri proanthocyanidin polymer composition can be enteric or non-enteric and can be SB-300 or SP 303. The dose and regimen of C. lechleri proanthocyanidin polymer composition administration are within the skill of the practitioner and will depend on the environmental conditions of the animals to be treated. In nonlimiting embodiments, it is envisioned that the animals can be prophylactically treated just after birth, e.g., days one to four, for from one to five days, or fewer, as necessary or desired.

In another of its aspects, the present invention provides the surprising result that treatment of non-human young animals with a Croton lechleri proanthocyanidin polymer composition, such as a C. lechleri botanical extract product, according to the invention provides one or more beneficial effects, for example, lower dehydration and higher fecal dry matter content, in treated animals that endures beyond the time period of actual administration of the treatment product to the animals, i.e., after cessation of the administration of the product to the animal. This unexpected carryover effect demonstrates that the administration of a C. lechleri proanthocyanidin polymer composition or C. lechleri botanical extract product, particularly an early administration to the young animal, followed by a period in which the product is not administered to the animal, may induce beneficial changes in the intestine of treated animals, i.e., a prebiotic effect, which is maintained beyond the actual course of the therapy. For example, an effect of early administration of C. lechleri proanthocyanidin polymer or C. lechleri botanical extract product, may endure for a time period greater than about one, two, three, four, or more weeks after treatment of the animal has ceased. In an aspect, early administration includes treating animals in need at the first sign of scours (diarrhea). In a particular aspect, the carryover effect may last for two to three weeks after cessation of treatment of the animal, thus allowing the animal to regain and maintain a healthy, normal gastrointestinal condition and function. In an aspect, the bacteria of the genera Faecalibacterium and Bifidobacterium, which comprise probiotic bacterial species, become more abundant as microbiota following treatment of diarrhea with an enteric coated C. lechleri derived proanthocyanidin product, e.g., a C. lechleri botanical extract product or SB-300.

Accordingly, the methods of the invention provide for periodic administration of the C. lechleri proanthocyanidin polymer or C. lechleri botanical extract product to an animal, such that an initial treatment may be given, followed by a time period, e.g., a lag of several days or even weeks, such as 1, 2, 3, 4, or more weeks, before another treatment, if any, is given.

Also provided is a method of treatment of a bovine calf suffering from, or at risk of developing, diarrhea and dehydration, in which the method further maintains normal fecal consistency (stool formation) and reduces or prevents dehydration in the calf following cessation of treatment. The method comprises orally administering to a preweaned calf a pharmaceutical composition comprising an enterically coated, aqueous soluble proanthocyanidin polymer from Croton lechleri or a Croton lechleri-derived botanical extract two times per day prior to a meal, for two to three days. In a particular embodiment, the calf is treated with the enterically coated, aqueous soluble proanthocyanidin polymer from Croton lechleri or a Croton lechleri-derived botanical extract two times a day for three days. In an embodiment, the calf is administered a Croton lechleri-derived botanical extract. In an embodiment, the neonatal animal is administered a bolus of the enterically coated, aqueous soluble proanthocyanidin polymer from Croton lechleri or a Croton lechleri-derived botanical extract. In an embodiment, dry fecal consistency, lack of dehydration and/or a healthy gastrointestinal microbiota of the neonatal non-human animal or the calf is maintained and sustained for at least two to three weeks following cessation of treatment with the pharmaceutical composition comprising an enterically coated, aqueous soluble proanthocyanidin polymer from Croton lechleri or the Croton lechleri-derived botanical extract.

The disclosed methods and C. lechleri-derived proanthocyanidin polymer and botanical extract products used in the methods provide several advantages in the treatment of diarrhea (scours) in neonatal, pre-weaned non-human animals, e.g., bovine calves. Such advantages include reduced medication and labor and veterinary costs, which result in earlier weaning of animals and heavier weaning weights. In addition, the treatment of young animals in accordance with the methods and products of the invention may also reduce the quantity of electrolytes used in standard of care to treat diarrhea-related dehydration and other symptoms, which is also of economic and commercial benefit.

In view of the evidence of the prebiotic effect of the C. lechleri proanthocyanidin polymer or botanical extract, the invention further provides methods of improving the intestinal microbiota, establishing a favorable intestinal microbiota and re-balancing the microbiota to favor beneficial microbes in any non-human animal by administering to the non-human animal a composition comprising a polyphenol-containing extract from a Croton species or Calophyllum species, particularly from C. lechleri, including the proanthocyanidin polymer composition or botanical extract from C. lechleri. The methods involve administration of polyphenol-rich compositions of the invention to non-human animals that do not have symptoms or diagnostic indicia of intestinal infection or disease to prevent intestinal disease or reduce the incidence and/or severity of intestinal disease, improve health and vitality, and/or increase weight gain. The methods further involve administration of a polyphenol-rich compositions of the invention to animals having an intestinal disease or disorder to facilitate treatment of the intestinal disease or disorder. The compositions of the invention may be administered for a short course of treatment, such as 1, 2 or 3 days or 1-10 days or 1-20 days or for one month to establish a beneficial intestinal microbiota in the non-human animal or may be administered chronically, either daily, weekly, or monthly to establish and/or maintain a beneficial intestinal microbiota in the non-human animal. The prebiotic formulation may be administered as a paste, gel, animal feed or medicinal feeding block formulation as further detailed herein. The prebiotic compositions of the invention may optionally be administered in combination with a probiotic containing beneficial microbes to promote and facilitate the development and establishment of a beneficial intestinal microbiota. The compositions of the invention having a prebiotic effect may be administered to non-human animals to promote normal stool formation and regularity and improve intestinal health in both healthy non-human animals and in non-human animals having some form of intestinal disease, such as but not limited to an intestinal infection. The non-human animal may be an adult, young animal or neonate. The composition may be administered as a prebiotic to livestock, race animals, companion animals, exotic animals, etc. The animals may be, for example, bovine, equine, ovine, porcine, fowl, camels, dogs, cats, rodents, etc. Example 6 herein describes an increase in abundance of prebiotic bacteria, namely, in the genera Bifidobacterium and Faecalibacterium, in non-human animals treated for diarrhea with a C. lechleri botanical extract product, such as enteric-coated SB-300.

In particular, provided is a method of inducing an intestinal microbiota favoring normal fecal consistency (stool formation) and diarrhea reduction or prevention in a neonatal, non-human animal suffering from diarrhea and its accompanying symptoms, in which the method comprises orally administering to the animal a pharmaceutical composition comprising an enterically coated, aqueous soluble proanthocyanidin polymer from Croton lechleri or a Croton lechleri-derived botanical extract rich in polyphenols at least once a day, preferably, prior to a meal, for at least two days. In a particular embodiment, the neonatal animal is treated with the enterically coated, aqueous soluble proanthocyanidin polymer from Croton lechleri or a Croton lechleri-derived botanical extract as two times a day for three days. In an embodiment, the neonatal animal is administered a Croton lechleri-derived botanical extract. In an embodiment, the neonatal animal is administered a bolus of the enterically coated, aqueous soluble proanthocyanidin polymer from Croton lechleri or a Croton lechleri-derived botanical extract.

In another aspect, the administration of a C. lechleri proanthocyanidin polymer, composition, or botanical extract to a non-human young or adult animal may provide an interactive or synergistic effect with the animal's intestinal microbiota profile or gut microbiome composition, which supplements and/or improves the animal's overall intestinal/gut health and heightens weight gain. In an embodiment, the animal is a non-human preweaned animal with diarrhea. In an embodiment, the animal is a non-human preweaned animal which is not afflicted with diarrhea. In an embodiment, the animal is a non-human adult animal with diarrhea. In an embodiment, the animal is a non-human adult animal which is not afflicted with diarrhea. In an embodiment, average daily weight gain is improved/increased and the fecal dry weight score is increased in animals treated with a C. lechleri proanthocyanidin polymer, composition, or botanical extract, e.g., SB-300 and enteric coated SB-300. In an embodiment, average daily weight gain is increased in young animals treated with a C. lechleri proanthocyanidin polymer, composition, or botanical extract, e.g., SB-300 and enteric coated SB-300 at 60 days of life. Without wishing to be bound by theory, the beneficial anti-secretory action resulting from treatment with C. lechleri proanthocyanidin polymer, composition, or botanical extract may act synergistically with the activity of a C. lechleri botanical extract, composition or polymer to increase, positively modulate, or advantageously alter the intestinal microbiome/microbiota profiles of a treated animal to provide an enhanced, augmented, improved, increased, or heightened effect against diarrhea and/or to improve the intestinal microbiome, thus contributing to and resulting in improved weight gain in the treated animals. The potential of the present methods involving the administration of a C. lechleri proanthocyanidin polymer, composition, or botanical extract to positively modulate, potentiate, or alter the gut microbiota and improve/increase weight gain and increase fecal dry matter in treated animals, such as dairy calves, may also beneficially influence the lifetime health and productivity of the treated animals.

### BRIEF DESCRIPTION OF THE FIGURES

FIG. 1 shows the effect of a treatment method involving the administration of proanthocyanidin polymer from Croton lechleri according to the invention on average fecal score number as described in Example 2 herein. Calves were scored twice daily for three days using a 3 level scoring system. As observed, the C. lechleri proanthocyanidin polymer (crofelemer)-treated calves demonstrated faster improvement on diarrhea scores starting on the second day after treatment. In FIG. 1, the average fecal score is shown on the y-axis and the number of treatment days is shown on the x-axis.

FIG. 2 shows results from a calf study in which young calves experiencing diarrhea and illness caused by enterogenic bacterial infection were treated with the C. lechleri proanthocyanidin polymer SB-300 in either enteric or reconstituted powder form, or with placebo. The graph presents fecal score (fecal consistency rating) versus day of treatment, as described in Example 3.

FIG. 3 presents results from the calf study of Example 3 in which young calves experiencing diarrhea and illness caused by enterogenic bacterial infection were treated with the C. lechleri proanthocyanidin polymer SB-300 in either enteric or reconstituted powder form, or with placebo. The percent of calves with watery diarrhea versus day of treatment is shown. A reduction in calf morbidity can be observed in the animals treated with C. lechleri proanthocyanidin polymer SB-300, in particular, the enteric form.

FIG. 4 presents results from the study of Example 3 and shows the effect of treatment on calves' fecal scores measured twice daily from pre-challenge (sample 1) to day 10 (sample 20). The bottom line on the graph (blue) represents calves in the group that received a bolus of enteric coated C. lechleri botanical extract containing proanthocyanidin (ECROF); the top line on the graph (red) represents calves in the control group (CTR) that received a placebo bolus. Error bars represent the standard error of the mean. The treatment period is represented by the horizontal bar (orange) in the lower left portion of the graph.

FIG. 5 presents results from the study of Example 3 and shows the effect of treatment on calves' fecal scores based on fecal consistency measured on fecal samples collected twice daily, averaged and summarized in the following categories: pre-challenge (first examination, baseline); during treatment (2^{nd} to 7^{th} examination); and after treatment (8^{th} to 24^{th} examination). Error bars represent the standard error of the mean. The right-most bars (blue) in each portion of the graph represent ECROF treated animals; the left-most bars (red) in each portion of the graph represent CTR animals. The different letters within the time frames depicted represent statistical differences (P-value <0.05).

FIG. 6 presents results from the study of Example 3 and shows the effects of treatment on calves' fecal dry matter content (percentage of dry matter) measured on fecal samples collected twice daily from pre-challenge (sample 1) to day 5 (sample 10). The top line (blue) represents the results from calves in the in the group that received a bolus of enteric coated C. lechleri botanical extract containing proanthocyanidin (ECROF); the bottom line on the graph (red) represents calves in the control group (CTR) that received a placebo bolus. Error bars represent the standard error of the mean.

FIG. 7 presents results from the study of Example 3 and shows dehydration scores obtained by scoring calves' skin turgor and eyes recession. Error bars represent the standard error of the mean. The right-most bars (blue) in each portion of the graph represent ECROF treated animals; the left-most bars (red) in each portion of the graph represent CTR animals. The different letters within the time frames depicted represent statistical differences (P-value <0.05).

FIG. 8 presents the relative abundance percent of the most prevalent Genera found in fecal samples of Control calves before challenge (Pre, 1). During treatment days (2 to 7) and after treatment cessation (8 to 24), relative abundance change was calculated using the relative abundance of each time point minus the initial (baseline) relative abundance.

FIG. 9 presents the relative abundance percent of the most prevalent Genera found in fecal samples of enteric coated SB-300-treated calves before challenge (Pre, 1). During treatment days (2 to 7) and after treatment cessation (8 to 24) relative abundance change was calculated using the relative abundance of each time point minus the initial (baseline) relative abundance.

FIGS. 10A-C: Data from the most relevant Genera after treatment cessation were averaged for each calf and correlation coefficient was calculated between calves in the control group (CTR) and calves treated with enteric-coated SB-300, (ECROF), (varying between −1 to +1). (A) Negative values were bacterial genera most highly associated with calves in the control group, while positive values were bacterial genera most highly associated with calves treated with enteric-coated SB-300. In addition, two relevant genera, namely, Faecalibacterium and Bifidobacterium, were used for individual comparison between treatment groups, (B, C).

### DESCRIPTION OF THE INVENTION

The invention provides treatment methods effective for reducing the incidence or severity of and/or alleviating diarrhea in neonatal, unweaned non-human animals in need thereof. In particular, the methods are directed to the treatment of diarrhea, particularly secretory/watery diarrhea, or episodic diarrhea, caused by a variety of etiological agents and/or environmental factors in neonatal and young (juvenile, non-adult) animals, particularly where scourges of diarrhea in such immature animals can have a profound economic impact for the animal agriculture, food and health industries. The invention further provides formulations and compositions suitable for treating diarrhea in neonatal and young animals. Unless otherwise noted herein, use of the term “animal” herein denotes non-human, warm-blooded mammals of a number of different species. In addition, the terms “young”, non-adult”, “immature” and “juvenile” are used synonymously herein and generally refer to animals under one year of age.

The invention further provides a prebiotic composition comprising polyphenol-containing extracts of Croton or Calophyllum species, particularly, C. lechleri, including SB-300, SP-303 and less purified botanical extracts of C. lechleri, that promote the growth of beneficial microorganisms in the gut of non-human animals to improve digestive health, treat, prevent, reduce the incidence and/or severity of diarrheal disease and other intestinal disease, increase weight gain and improve vitality and reduce mortality and morbidity within a veterinary population. The invention also, thus, provides methods of promoting growth of beneficial microorganisms, establishing or re-establishing a beneficial intestinal microbiota, and improving intestinal health by administering to a non-human animal a prebiotic composition containing an extract of a Croton or Calophyllum species, particularly, C. lechleri. Such beneficial organisms include, but are not limited to, Lactobacillus, Bifidobacterium, Faecalibacterium, and Saccharomyces. The animal may be a neonate, a juvenile or young animal, or an adult animal. The prebiotic composition may be administered to any non-human animal, particularly, grazing animals and livestock, such as cows, sheep, goats, pigs, bison and buffalo, etc., fowl, racing animals, such as horses and camels, companion animals, such as dogs, cats and rodents, and exotic animals. The animals may not exhibit symptoms of an intestinal disease or disorder or may suffer from one or more intestinal diseases or disorders. In specific embodiments, the prebiotic composition is administered along with a probiotic composition containing microorganisms beneficial to the intestine and the growth of which is promoted by the prebiotic composition of the invention.

The methods of the invention provide a solution to a significant need for the animal industry, e.g., the beef and dairy industries worldwide, in which neonatal calf diarrhea presents one of the largest health challenges, as well as economic losses. In addition, the methods of the invention provide a solution to the common problem of watery diarrhea, including episodic diarrhea, in horse foals. The methods and treatments of the invention improve gastrointestinal/gut health and normalize stool formation in young animals suffering from diarrheal conditions, including, by way of example, watery diarrhea in horse foals and bovine calves. The methods and treatments of the invention further improve gastrointestinal/gut health in non-human animals, including neonates, young animals and adults, by promoting and maintaining a beneficial intestinal microbiota.

The methods and treatments of the invention are particularly suitable for treating animals of a young age. In an embodiment, the animals are neonatal (or newborn), unweaned, non-adult animals that are born, bred, raised and/or maintained in a domesticated and/or agricultural setting, e.g., as livestock and farm animals, for commodities such as food, labor, sport, or other commercial or non-commercial agricultural husbandry capacity. Nonlimiting examples of animals affected by diarrhea and treatable by the methods and formulations of the invention include, without limitation, neonatal and young cattle (calves), young bison or buffalo, pigs (piglets), sheep (lambs), goats (kids), horses (foals) and camels (calves), as further described herein. In an embodiment, the neonatal or young animals are domestic, companion animals, such as, without limitation, dogs and cats of any species. As used herein, the terms “neonatal” and “newborn” are synonymous and generally refer to animals two weeks of age or less.

The present invention relates to treating diarrhea in neonatal, unweaned and young animals with physiologically and pharmaceutically acceptable formulations and compositions comprising a therapeutically effective amount of an antidiarrheal agent comprising a proanthocyanidin polymer obtained from a Croton spp., preferably Croton lechleri. The proanthocyanidin polymer composition can also be obtained from a Calophyllum spp., in particular Calophyllum inophylum. In an specific embodiment, the pharmaceutically acceptable composition comprises a proanthocyanidin polymer from Croton lechleri. In an specific embodiment, the pharmaceutically acceptable composition comprises a botanical extract derived from Croton lechleri that is rich in polyphenols.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention.

According to the invention, treatment of an animal in need thereof typically involves the use or administration of an effective amount or a therapeutically effective amount of a proanthocyanidin polymer or a proanthocyanidin polymer composition or prebiotic composition, preferably from a Croton spp., particularly C. lechleri, provided as either an enteric or non-enteric formulation. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods are directed to ameliorating, preventing, inhibiting, reversing, attenuating, alleviating, abrogating, minimizing, suppressing, reducing, decreasing, diminishing, stabilizing, eradicating, curing, or eliminating diarrhea and/or its associated symptoms caused by a variety of different agents or environmental factors and influences that adversely affect the health, growth and survivability of neonatal and young animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are suitable for treating diarrhea resulting from different mechanisms involved in the pathogenesis of the disorders, for example, osmotic diarrhea, secretory diarrhea, episodic diarrhea, or inflammatory and infectious diarrhea. In an embodiment, the neonatal or young animal can suffer from diarrhea associated with inflammation of the lining of the colon, such as colitis, or acute colitis, which can be caused by infection or inflammation of the bowel.

Osmotic diarrhea is associated with absorption of water in the intestines, which depends upon adequate absorption of solutes. If excessive amounts of solutes are retained in the intestinal lumen, water will not be absorbed and diarrhea results. Osmotic diarrhea typically results from ingestion of a poorly absorbed substrate, for example, a carbohydrate or divalent ion or from malabsorption of any type, such as an inability to absorb certain carbohydrates. Secretory diarrhea occurs when the secretion of water into the lumen of the intestine exceed absorption. Under normal conditions, large volumes of water are secreted into the small intestinal lumen, but a large portion of this water is efficiently absorbed before reaching the large intestine.

Secretory diarrhea can result from exposure of an animal to toxins (enterotoxins) from certain types of bacteria, such as cholera toxin of Vibrio cholerae and heat-labile toxin of E. coli. Massive diarrhea is induced from such microorganisms as a consequence of their toxins strongly activating adenylyl cyclase, which causes a prolonged increase in the intracellular concentration of cyclic AMP within crypt enterocytes. This increase, in turn, results in prolonged opening of the chloride channels that contributes to secretion of water from the crypts, thereby allowing uncontrolled secretion of water. These bacterial toxins can also affect the enteric nervous system, resulting in an independent stimulus of water secretion.

Inflammatory and infectious diarrhea can be caused by the disruption of the epithelium of the intestine due to microbial or viral pathogens. Typically, the epithelium of the digestive tube is protected from insult by a number of mechanisms that constitute the gastrointestinal barrier. However, the gastrointestinal barrier can be breached and result in diarrhea. Destruction of the epithelium results not only in leaking of serum and blood into the lumen but also is often associated with significant destruction of adsorptive epithelium. When this occurs, the absorption of water becomes highly inefficient and diarrhea results. The pathogenic culprits frequently associated with infectious diarrhea include bacteria, such as E. coli, Campylobacter and Salmonella; viruses, such as rotaviruses, coronaviruses, parvoviruses and norovirus; and protozoa, such as coccidia species, Cryptosporium and Giardia. In addition, the response of the immune system to inflammatory conditions in the bowel contributes greatly to the development of diarrhea. Activated white blood cells are stimulated to produce and secrete inflammatory mediators and cytokines that stimulate secretion. An secretory component is thus imposed upon and exacerbates an inflammatory diarrhea. Moreover, reactive oxygen species produced by leukocytes can damage or destroy intestinal epithelial cells, which are replaced with immature cells that are generally lacking in the brush border enzymes and transporters necessary for the absorption of nutrients and water. Thus, components of an osmotic (malabsorption) diarrhea provide additional pathology and problems for an afflicted animal.

Thus, in various embodiments, the diarrhea to be treated is caused by infection or invasion of the animals by pathogens, including bacteria, e.g., Escherichia coli, Salmonella spp., Clostridium perfringens, etc.; viruses, e.g., coronaviruses, rotaviruses, bovine virus diarrhea (BVD) virus, infectious bovine rhinotracheitis (IBR) virus, etc.; protozoa, e.g., Cryptosporidium, coccidia, etc.; as well as yeasts and molds. In some cases, diarrhea can be caused by a single infectious microorganism; however, mixed infections, such as caused by, e.g., E. coli plus Cryptosporidium, or Coronavirus plus Salmonella spp., are also not uncommon.

The gram-negative bacterium Escherichia coli is normally found in the intestines of most animals. Although most E. coli are nonpathogenic, some are able to cause intestinal and extraintestinal infections. Large numbers of E. coli are present in the farm environment as a result of fecal contamination. Initial exposure to pathogenic E. coli may occur in contaminated calving pens, but systemic infection usually requires predisposing environmental factors, inadequate transfer of passive immunity or compromised immune system by other infection. The most common type of colibacillosis in young animals is caused by the non-invasive Enterotoxigenic E. coli (ETEC) strains, e.g., K99 STa, which are also the leading cause of diarrhea among travelers and children in the developing world (B. Nagy and P. Z. Fekete, 2005, Int J Med Microbiol., 295:443-454).

Treatment of neonatal and young animals according to the methods of the invention is of particular importance, because such immature animals are most susceptible to infection by numerous pathogens of many types; resistance to infection develops with increasing age of the animal. In addition, younger animals experience more severe clinical illness as a result of infection and resulting diarrhea. For example, young animals (lambs) at one to five days of age experienced more severe infection by enteric cryptospores, causing protracted diarrhea, wasting and death, while young lambs at thirty days of age, which had become infected, did not exhibit severe signs of clinical disease. In general, due to the anatomy of the gastrointestinal tract of adult animals such as horses, conditions affecting the large intestine and cecum typically cause diarrhea. However, young animals, e.g., foals, that are less than about three months of age do not have fully competent large intestines and ceca as do adult animals; therefore, young animals tend to be more prone to diarrhea caused by small intestinal conditions. In general terms, a foal is an equine, particularly a horse, that is one year old or younger in age.

In an embodiment, the diarrhea to be treated results from noninfectious causes, for example, without limitation, inadequate nutrition and/or insufficient attention of the neonate or young animal on the part of the mother, exposure to severe environment, or a combination of these events. In another embodiment, diarrhea results from a combination of the invasion of infectious microorganisms and noninfectious factors. Frequently, noninfectious causes of diarrhea in young animals are considered to be factors that predispose or contribute to an animal's susceptibility to infectious agents and causes of diarrhea. Whether the cause of diarrhea in animals is infectious or noninfectious, the absorption of fluids from the intestine is altered and life-threatening electrolyte imbalances can occur. The affected animals lose fluids, rapidly dehydrate and suffer from electrolyte loss and acidosis. Although infectious agents may cause an initial damage to the animal's intestine, actual death from diarrhea (serious diarrhea) in animals usually is a consequence of dehydration, acidosis and loss of electrolytes, which may be difficult to replenish in adequate amount and time. Accordingly, the methods and formulations of the invention are suitable for treating diarrhea and the symptoms of diarrhea, such as dehydration, weight loss, and electrolyte loss, in an effort to prevent more severe dehydration and animal death.

Because newborn non-human animals, such as calves, are born without a yet functional immune system and without most antibodies that can fight the infectious agents causing sickness and diarrhea in these immature animals, vigilant attention should be paid to the conditions and health of these young animals, particularly during inclement or severe weather conditions and/or difficult births. Neonatal and young animals acquire antibodies from colostrum, which is optimally received by the animals before they are two to four hours old. As young animals grow older, they rapidly lose their ability to absorb colostral antibodies. Thus, for example, colostrum provided to calves that are more than 24 to 36 hours old will likely not be effective, as antibodies are infrequently absorbed following this time in the animal's life.

Due to the unique physiology and susceptibility to diarrheal disease of neonatal animals, prophylactic administration of the C. lechleri proanthocyanidin polymer composition can reduce the incidence of diarrheal disease in neonatal animals, improving health, weight gain and survivability in populations of neonatal animals.

In an embodiment, treatment of non-human animals, such as preweaned calves, with a C. lechleri proanthocyanidin polymer, composition, or botanical extract according to the present methods provides a beneficial effect in increasing weight gain and in supporting a diverse and healthy gut microbiome in the animal throughout the animal's lifetime. In a particular embodiment, the treatment methods involving the administration of a C. lechleri proanthocyanidin polymer, composition, or botanical extract to neonatal calves having diarrhea, e.g., afflicted with scours, support weight gain in preweaned calves at about 10 days of life to about 60 days of life, and potentially longer. In another embodiment, the treatment of animals, especially preweaned animals and animals in the early weeks of life, e.g., from two to eight weeks of life, with a C. lechleri proanthocyanidin polymer, composition, or botanical extract, e.g., enteric SB-300, may increase fecal microbial diversity in the treated animals, which may result in a corresponding increase in weight gain during the pre-weaning period. In another embodiment, in addition to the anti-secretory effects afforded by the methods herein, treatment of animals with a C. lechleri proanthocyanidin polymer, composition, or botanical extract according to the present methods may supplement or synergize with an alteration in the intestinal microbiota profiles of the treated animals, leading to heightened and positive improvements in the animals' gut microbiome profile and/or composition, thus contributing to the animals' overall health and improved and/or increased weight gain.

Increased fecal microbial diversity in animals treated with a C. lechleri proanthocyanidin polymer, composition, or botanical extract according to the present methods is likely to reflect a diversity of gut microbiota of the animals, which is physiologically appropriate, thus leading to overall intestinal health and lasting improvement in the animals' intestinal microbiota profiles. As would be appreciated by the skilled practitioner, the gut microbiota of dairy calves influences major aspects of the animal's postnatal life, such as the development of the immune system, which may potentially alter and improve the animals' physiology. A low incidence of diseases and efficient growth in preweaned dairy calves are important for optimal post-weaning performance. In addition, the average daily weight gain may influence lifetime productivity for non-human animals, such as dairy cattle, because pre-weaning nutrition can have a significant effect on mammary gland development, the timing of puberty and the age at which a dairy cow first produces milk. Thus, in an embodiment, a C. lechleri proanthocyanidin polymer, composition, or botanical extract is administered to a neonatal, preweaned animal with a diarrheic condition, e.g., scours, to improve weight gain and to provide a gut microbiota that is conducive to and supports the prolonged intestinal health of the animal, e.g., during the life of the animal after the weaning period. In another embodiment, a C. lechleri proanthocyanidin polymer, composition, or botanical extract is administered to a neonatal, preweaned animal without a diarrheic condition to contribute positively to weight gain and to provide or supplement a gut microbiota in the animal that is conducive to and supports the prolonged intestinal health of the animal, e.g., during the life of the animal after the weaning period.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. See also, Ubillas et al., 1994, Phytomedicine, 1:77. The isolation of an aqueous soluble proanthocyanidin polymer composition from Calophyllum inophylum and the use of this composition as an antiviral agent are also described in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

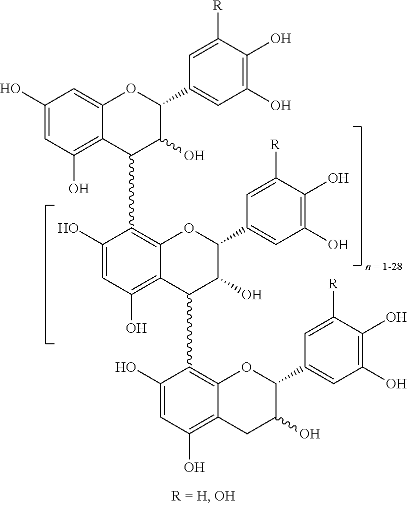

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin, (−)-epicatechin and (+)-catechin and is suitable for use in the enteric and non-enteric formulations and compositions for administration in the treatment methods described herein. The C. lechleri proanthocyanidin may also be isolated according to example 2 of patent application publication US2007/0254050 or in patent application publication US2005/0019389, which are both incorporated by reference herein in their entirety.

In another embodiment, SB-300 is the proanthocyanidin polymer from Croton lechleri, or a pharmaceutically acceptable formulation or composition comprising SB-300, which is suitable for use in the treatment methods of the invention. SB-300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated by reference herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7. Such extracts can be rich in polyphenols and have beneficial antioxidant and prebiotic properties.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below). In another embodiment, the proanthocyanidin polymer is formulation without an enteric coating or matrix. Both enteric and non-enteric forms of the proanthocyanidin polymer from Croton lechleri, for example, SB-300, are intended for use in the methods of the present invention.

### Preparation of Proanthocyanidin Polymer Compositions and Formulations

The proanthocyanidin polymer composition, effective for treating secretory diarrhea according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophyllum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated by reference herein. PCT application PCT/US00/02687, published as WO 00/47062, the contents of which are incorporated by reference herein, also discloses a method of manufacturing a proanthocyanidin polymeric composition isolated from Croton spp. or Calophyllum spp., and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB-300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Methods of Treatment and Applications of Use

The invention is directed to methods of treating diarrhea associated with pathogenic infection and non-pathogenic causes, particularly in neonatal and young animals, comprising administering to an animal in need of such treatment, a pharmaceutically acceptable composition comprising a proanthocyanidin polymer from a Croton species or Calophyllum species in an amount effective to treat the diarrhea. In preferred embodiments, the proanthocyanidin polymer is from a Croton species, namely, Croton lechleri. Treating the diarrhea can involve reducing the severity and duration of the diarrhea in the animal. Treating the diarrhea can also involve increasing the survivability, vigor and weight of the animal, particularly a neonatal or young animal undergoing treatment. In an embodiment, the diarrhea is secretory or watery diarrhea.

The methods of the invention relate to the treatment of non-human animals, notably, but not limited to, the newborns and young of livestock, domestic and farm animals, including grazing animals, which are oftentimes relatively large in size. In one embodiment, the immature animals to which treatment with the proanthocyanidin polymer from Croton lechleri is administered are neonatal (newborn) or infant animals, for example, one to ten hours after birth, one to fifteen hours after birth, twelve to twenty-four hours after birth, twenty-four to thirty-six hours after birth, one to three days after birth, one to four days after birth, one to six days after birth, or one to seven days after birth or up to two weeks after birth. Neonatal animals generally being those under two weeks of age. In an embodiment, the animals are treated between day one and day four after birth. In some embodiments, the neonatal or young animals are treated one to five days of age, less than one week of age, or only a few weeks of age. In an embodiment, treatment occurs during the first weeks of life, for example, one to six weeks of age. In an embodiment, the animals are from two to ten weeks of age, for example, less than one, two, three, four, five, six, seven, eight, nine, or ten weeks of age. The animals undergoing treatment may also be from one to four weeks of age, from one to six weeks of age, or from two to four weeks of age. In some embodiments, the animals are one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days old. In other embodiments, the animals are thirty to forty days old. In other embodiments, the animals are young animals, generally up to one year in age. In many cases, the animals are not weaned (unweaned), i.e., they are still drinking milk. For example, dairy calves are generally weaned at 60-80 days while beef cattle may be weaned at 3-8 months of age, pigs at 3 weeks of age, dogs at 7-8 weeks, and horses at 4-6 months of age. Also in many cases, neonatal is synonymous with unweaned. In some cases, the animals are newly weaned or weaned, but still juvenile, young, and non-adult. Such young animals are also highly susceptible to becoming afflicted with diarrhea from various infectious and/or environmental causes.

According to the methods of the invention, the neonatal and young animals can be treated with a proanthocyanidin polymer from C. lechleri, e.g., SB-300, or a botanical extract derived from C. lechleri, for one, two, three, four, five, six, seven, eight, nine, or ten days, etc. The C. lechleri proanthocyanidin polymer can be administered to the animal on consecutive days or intermittently, such as every other day, every two days, every three days, every four days, and the like. In an embodiment, the C. lechleri proanthocyanidin polymer is administered to the animals for three consecutive days. In embodiments, the C. lechleri proanthocyanidin polymer is administered to the animals twice daily, three times daily, or four times daily, for three consecutive days. In an embodiment, the C. lechleri proanthocyanidin polymer is administered to neonatal animals between one and four days after birth for three consecutive days. As understood by the skilled practitioner, environmental, e.g., farm, conditions surrounding the neonatal and young animals may dictate the start and course of a treatment regimen such that the administration of the C. lechleri proanthocyanidin polymer occurs earlier in the animal's life and for a longer duration, especially since diarrheal disease typically affects neonatal and young animals in about the first seven days of life, or between about day one or day four of life. In the foregoing embodiments, the animals are bovine or camel calves.

In a particular embodiment, a formulation or composition comprising a botanical extract derived from C. lechleri, SB-300, or SP 303, is provided in the form of a gel or paste formulation that is orally administered to the neonatal or young animal, such as a horse foal, or alternatively to adult animals, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of neonatal and young animals, such as horse foals, and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB-300 or SP 303 and is orally administered to animals. In a particular embodiment, the paste comprising SB-300 enteric beads is orally administered to an animal, such as a foal, in need twice daily for three days. In another particular embodiment, the paste comprising SB-300 enteric beads is orally administered to an animal, such as a foal, in need four times daily for three days. In some embodiments, the paste is orally administered for three consecutive days. In a particular embodiment, the paste comprising SB-300 enteric beads is orally administered at a dose of 2 mg/kg twice daily for three days, or three times daily, or four times daily for three days. The formulation is especially suitable for the normalization of stool formation in a short time period, e.g., less than a week or less than two weeks; for mitigation of weight loss; and reduction in supportive care costs, rehydration therapies, such as oral rehydration, in a young animal, or, in an adult, afflicted with diarrhea and undergoing treatment.

In other embodiments, the proanthocyanidin polymer composition or prebiotic composition of the invention is administered to animals, particularly ruminant livestock, by a medicated feeding block. A medicated block is a compressed feed material that contains the composition of the invention, and is commonly packaged in a cardboard box for feeding to livestock. Animals have free access to the block and, thus, should be used when precise dosage is not of concern. The amount of active ingredient ingested by the animal may be regulated by altering the formulation to alter the palatability and/or the hardness of the medicated block. For example, molasses increases palatability and sodium chloride decreases it. Additionally, the incorporation of a binder such as lignin sulfonate in blocks manufactured by compression or magnesium oxide in blocks manufactured by chemical reaction, increases hardness. The hygroscopic nature of molasses in a formulation may also impact the hardness of medicated blocks and is addressed by using appropriate packaging. A medicated feeding block may be particularly suited to administration of the prebiotic compositions of the invention.

The types of non-human animals for which the treatment methods are suitable are not particularly limited as to animal type, genus, or species. In general, neonatal or young farm animals, food-source animals, livestock animals, animals bred or kept for various purposes, such as sport (e.g., racing, riding), transport, domestic, companion, industrial uses (e.g. hauling, pulling, plowing), and the like, are particularly amenable to treatment according to the methods of the invention. For example, encompassed by the methods of the invention is the treatment of neonatal or young non-human animals or adult animals, such as cows (calves), cattle or steer (calves), camels (calves), rams and sheep (lambs), horses (foals), pigs (piglets), goats (kids), bison/buffalo (calves), llamas, donkeys, mules, yaks, etc. Neonatal or young exotic animals or adult animals, such as zoo animals of various species, are also embraced by the treatments of the invention. In an embodiment, the animals are grazing animals. The treatment of diarrhea in neonates and unweaned animals, for example, calves (bovine, camel, buffalo/bison), lambs, piglets, and foals (equine) is particularly embraced by the described methods.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutically acceptable formulations and methods of the invention are useful in prophylactic and therapeutic applications in the treatment of secretory diarrhea, especially in preventing the dehydration and electrolyte loss that accompanies secretory/watery diarrhea.

In a particular embodiment, the methods of the invention treat diarrhea resulting from infection by the Salmonella spp. microorganism with an effective amount of a polymeric proanthocyanidin polymer composition from a Croton species or Calophyllum species, or with a latex, extract or food supplement botanical extract derived therefrom. The treatment of diarrhea caused by Salmonella spp. with a proanthocyanidin polymer composition from Croton lechleri, or with a latex, extract or food supplement botanical extract derived therefrom is an unexpected and surprising aspect of the invention, because Salmonella spp. cause diarrhea by a mechanism of action and by affecting cellular pathways and responses that is distinct and different from the mechanism of action associated with the activity of proanthocyanidin polymer compositions.

More specifically, mechanism of action of polymeric proanthocyanidin polymer compositions, e.g., crofelemer, is through the inhibition of both the cystic fibrosis transmembrane conductance regulator protein (CFTR) chloride ion channel and the calcium-activated chloride ion channels (CaCC). The polymeric proanthocyanidin polymer composition acts by blocking chloride ion channel secretion and the accompanying high volume water loss occurring in diarrhea, thus normalizing the flow of chloride ions and water in the gastrointestinal (GI) tract. However, Salmonella microorganisms trigger diarrhea in infected hosts by producing several virulence factors. One such factor is a protein called SopE, which is injected into intestinal epithelium cells where it triggers a cascade of intracellular signaling events once the bacteria enter the GI tract. (See, e.g., S. Zhang et al., 2003, Infection and Immunity, 71(1):1-12; and A. J. Mueller et al., 2009, Cell Host and Microbe, 6(2):125-136). The binding of the SopE protein to two specific GTPase proteins alters the cell membrane and allows the bacteria to penetrate the cell. In addition, the two GTPase proteins activate Caspase-1 inside the cell, which is a key factor in inflammatory responses. Caspase-1, in turn, causes the production of proinflammatory mediators (cytokines) that attract macrophages which phagocytize the bacteria that has penetrated into the intestinal tissue and cells; however, Salmonella bacteria remaining in the intestinal lumen are not seriously affected. The heightened immune response that exists in the infected animals as a consequence of the infection results in serious inflammation, fluid accumulation and distress for the host animal.

Because Salmonella, which causes a disease pathology and an inflammatory immune response that lead to diarrhea without significantly affecting the CTRF or CaCC, it is considered quite surprising and unexpected that a proanthocyanidin polymer composition which functions by inhibiting these channels is effective in treating diarrhea induced by the Salmonella microorganism. However, the treatment of diarrhea in Salmonella-infected neonatal and young animals, such as, e.g., bovine calves and piglets, with a proanthocyanidin polymer composition (e.g., SB-300) according to present methods demonstrates an unpredicted effectiveness of the composition against diarrhea resulting from a source associated with a different etiology.

In an embodiment, the young animals treated by the methods of the invention are two to four weeks of age. In an embodiment, the animals are two to four week old calves, e.g., without limitation, bovine or camel calves, having diarrhea caused by infection with Salmonella, or crytosporidia or a combination thereof. In an embodiment, the animals are two to four week old calves, e.g., without limitation, bovine or camel calves, having undifferentiated diarrhea of unknown origin. In an embodiment, the animals are horse foals suffering from diarrhea associated with certain adverse environmental conditions and/or infection. In other embodiments, the animals treated by the methods of the invention are approximately 3-1000 kg in weight; or approximately 5-900 kg in weight, or approximately 10-350 kg in weight; or approximately 15-150 kg in weight; or approximately 25-60 kg in weight, or approximately 30-50 kg in weight, or approximately 30-40 kg in weight. In an particular embodiment, the young animal being treated for diarrhea is a bovine calf of approximately 20-40 kg in weight. In an particular embodiment, the young animal being treated for diarrhea is a camel calf of approximately 30-50 kg in weight.

In an embodiment, neonatal and young animals are treated prophylactically with a C. lechleri proanthocyanidin polymer composition, such as SB-300 or SP 303, in enterically protected or non-enterically protected form, to prevent or reduce the risk or severity of the debilitating effects of diarrheal disease and its associated symptoms, e.g., dehydration and weight loss, in neonatal and young animals. According to the treatment method, a C. lechleri proanthocyanidin polymer composition is administered to neonatal and young animals at a suitable time after birth to protect the animals from diarrhea outbreaks typically caused by infections and adverse environmental conditions. Administering a C. lechleri proanthocyanidin polymer composition to neonatal and young animals can also serve to ameliorate or reduce the risk of the animals' suffering from a more serious or severe form of diarrhea relative to animals that are not provided with the C. lechleri proanthocyanidin polymer composition prior to an outbreak of disease or infection. The C. lechleri proanthocyanidin polymer composition can be enteric or non-enteric and can be, for example, SB-300 or SP 303. The dose and regimen of C. lechleri proanthocyanidin polymer composition administration are within the skill of the practitioner to determine and will depend on the environmental conditions and health of the neonatal and young animals to be treated. The animals can be prophylactically treated a with C. lechleri proanthocyanidin polymer composition according to the invention, for example and without limitation, one to seven days, one to six days, one to four days, one to three days, or one or two days after birth. The treatment regimen can involve one, two, three, four, five, six, seven or more days, of C. lechleri proanthocyanidin polymer composition administration to the animals, modified or adjusted as necessary or desired, once or multiple times, e.g., twice, three or four times, per day. The animals can be regularly observed and monitored for health improvements and weight gain. These prophylactic methods of the invention can improve weight gain within the first 15, 20, 25 or 30 days by at least 5%, at least 10%, at least 15%, or even at least 20%.

### Prebiotic Compositions and Methods

In view of the evidence of the prebiotic effect of the C. lechleri proanthocyanidin polymer or botanical extract, the invention further provides methods of improving the intestinal microbiota, establishing a favorable intestinal microbiota and re-balancing the microbiota to favor beneficial microbes in any non-human animal by administering a prebiotic composition comprising a polyphenol-containing extract of a Croton or Calophyllum species, particularly, C. lechleri, and including a C. lechleri proanthocyanidin polymer composition or polyphenol-containing C. lechleri botanical extract of the invention. The methods involve administration of the compositions to non-human animals that do not have symptoms or diagnostic indicia of intestinal infection or disease to prevent or reduce the incidence or severity of intestinal disease, improve health and vitality, and/or increase weight gain. The prebiotic compositions may also be administered to non-human animals having one or more intestinal diseases or disorders to promote and facilitate treatment of the intestinal disease or disorder, including diarrheal diseases, inflammatory intestinal diseases, intestinal distress, etc. The compositions of the invention may be administered for a short course of treatment, such as 1-10 days or 1-20 days or for one month to establish a beneficial intestinal microbiota in the non-human animal or may be administered chronically, either daily, weekly, or monthly to establish and/or maintain a beneficial intestinal microbiota in the non-human animal. In one embodiment, the prebiotic compositions are provided ad libitum, for example, as a medicinal feed block. The compositions of the invention may optionally be administered in combination with a probiotic containing beneficial microbes to promote and facilitate the development and establishment of a beneficial intestinal microbial population. The compositions of the invention having a prebiotic effect may be administered to non-human animals to promote normal stool formation and regularity and improve intestinal health in both healthy non-human animals and in non-human animals having some form of intestinal disease, such as but not limited to an intestinal infection. The non-human animal may be an adult, young animal or neonate. The composition may be administered as a prebiotic to livestock, race animals, companion animals, exotic animals, etc. The animals may be, for example, bovine, equine, ovine, porcine, fowl, camels, dogs, cats, rodents, etc.

According to the methods of the invention, the non-human animals can be treated with a polyphenol containing extract of the invention, preferably from C. lechleri, including a proanthocyanidin polymer composition from C. lechleri, e.g., SB-300, or a botanical extract derived from C. lechleri, for one, two, three, four, five, six, seven, eight, nine, or ten days, etc. The C. lechleri proanthocyanidin polymer can be administered to the animal on consecutive days or intermittently, such as every other day, every two days, every three days, every four days, and the like. In an embodiment, the composition is administered to the animals for three consecutive days. In an embodiment, the composition is administered to neonatal animals between one and four days after birth for three consecutive days. In certain embodiments, the prebiotic composition of the invention is administered to maintain beneficial intestinal microbiota of a non-human animal and, as such, may be administered chronically and/or periodically to the animal. For example, the prebiotic composition may be administered daily, weekly, or monthly, or at an appropriate frequency to maintain the beneficial intestinal microbiota.

The prebiotic composition may be administered in any convenient form, including as a paste, gel, or bolus or in a feed block as described herein. The prebiotic composition may be administered in animal feed, as an animal feed composition, in a milk replacer, or other form for oral administration that will be acceptable to the animal to encourage consumption.

The prebiotic compositions of the invention may be administered in combination with any other agent that promotes the establishment and maintenance of a beneficial intestinal microbiota. For example, the prebiotic compositions of the invention may be administered with a probiotic composition containing beneficial microbes the growth of which in the intestine may be promoted or maintained by the prebiotic composition of the invention. Such organisms include, but are not limited to, Lactobacillus, Bifidobacterium, Faecalibacterium and Saccharomyces species.

### Physiologically and Pharmaceutically Acceptable Formulations

The invention provides formulations of proanthocyanidin polymer compositions and polyphenol containing botanical extracts from Croton and Calophyllum species, particularly, C. lechleri. The proanthocyanidin polymer composition or polyphenol-containing extract can be provided in any physiologically, pharmaceutically, or therapeutically acceptable form. The pharmaceutically acceptable composition can be formulated for oral administration as, illustratively, but without limitation, powders; crystals; granules; small particles, including particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. The pharmaceutically acceptable composition can also be formulated for oral administration as a solution or suspension in an aqueous liquid, as a liquid incorporated into a gel capsule, as a gel, as a paste or gel paste, or as any other convenient formulation for administration. The composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

The pharmaceutically acceptable formulation can also include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments, the pharmaceutically acceptable formulations contain the proanthocyanidin polymer composition with an enteric coating, in addition to another pharmaceutically acceptable vehicle. In an embodiment, the proanthocyanidin polymer composition can be directly-compressed into a tablet. The tablet can be without excipients and of pharmaceutically acceptable hardness and friability, optionally, with a lubricant, e.g., without limitation, magnesium stearate, and enteric coated. In another embodiment, the pharmaceutically acceptable compositions containing the proanthocyanidin polymer composition alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V Allen, Jr., Pharmaceutically acceptable Press, Royal Pharmaceutically acceptable Society, London, UK, 2013; and U.S. Pat. No. 7,323,195).

In an embodiment, the proanthocyanidin polymer composition is formulated with a substance that protects the proanthocyanidin polymer and/or the polymer composition from the stomach environment. For such protection, the proanthocyanidin polymer composition can be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5-2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food, particularly for administration to neonatal or young animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the proanthocyanidin polymer composition or prebiotic composition is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxy-propylmethylcellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition or prebiotic composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In an embodiment, the proanthocyanidin polymer composition or prebiotic composition is a gel or gel formulation. In an embodiment, the proanthocyanidin polymer composition or prebiotic composition is a paste formulation. In an embodiment, the paste formulation contains a purified botanical extract derived from C. lechleri. In another embodiment, the paste formulation contains enterically coated beads comprising SB-300 or SP 303. In an embodiment, the paste formulation contains enteric protected SB-300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to neonatal and young foals, but also is applicable for other adult and neonatal animals, such as those described herein. In an embodiment, the gel or paste is not contained in a delivery device, but is administered to the roof of the mouth of the animal, particularly one that is too incapacitated or ill to eat or drink, thereby eschewing an oral or other mode of administration. In an embodiment, the gel or paste comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the C. lechleri botanical extract, proanthocyanidin polymer composition, or a compressible form thereof are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In another embodiment, the proanthocyanidin polymer composition or prebiotic composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent.

Oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions or prebiotic compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer or prebiotic composition may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In a particular embodiment for treating diarrhea in neonatal animals, e.g., without limitation, bovine and camel calves, foals, kids, lambs, etc., the proanthocyanidin polymer composition or prebiotic composition is in powder, e.g., reconstitutable powder, form. The composition may be enterically coated or not enterically coated. In an embodiment, the neonates are less than one week in age. In an embodiment, the neonatal animals are bovine calves or camel calves. In an embodiment, the neonatal animals are afflicted with E. coli-induced secretory diarrhea. In an embodiment, the E. coli causative agent is E. coli K99 Sta. In an embodiment, in addition to infection with E. coli, the animal experiences involvement of a viral infection by rotavirus and/or coronavirus, whose mechanism of action involves infection and subsequent destruction of the cells lining the intestinal tract. Such cells are involved in the digestion and absorption of milk in the animal's gut. By treating diarrhea and associated dehydration in neonatal animals and allowing the animals to survive, the methods of the invention also provide the means for the cellular damage in the intestines of the treated neonates and young animals to be repaired.

In an embodiment, the powder form of the proanthocyanidin polymer composition or prebiotic composition used for treatment is reconstituted or mixed with liquid, such as oral electrolytes, milk or a milk replacer, water, physiological saline, to produce a liquid form or suspension. Milk replacer is generally a source of protein from different origins (for example, milk from a different species, soy, or eggs) and energy (lactose and fat) given to the calf or other animals to replace milk from the mother. In a specific embodiment, the composition is mixed at 200-800 mg per kg of the powder milk replacer prior to reconstitution. In an embodiment, the powder form of the composition is provided in the form of individual dosages in packets, e.g., packaged dosage forms, wherein some number of individual packets are provided for use in a treatment regimen. In certain embodiments, the packaged dosage form contain 50-500 mg of the proanthocyanidin polymer composition or polyphenol-containing extract, preferably, 200-300 mg of the proanthocyanidin polymer composition or polyphenol-containing extract. The number of individual doses that can be packaged and provided together is not intended to be limiting, and can include, for example, one to twenty packaged doses; one to ten packaged doses; two, four, six, eight, ten, or more packaged doses, as well as numbers of packaged doses in-between the foregoing, for efficiency of use, handling and for commercial efficacy. Those skilled in the art will appreciate that due to the higher purity of compositions such as SP-303 or crofelemer and SB-300, more by weight of SB-300 than SP-303 will need to be used in formulations to achieve the same amount of the active ingredient of the proanthocyanidin polymer composition. SB-300 generally has about 67% by weight of the proanthocyanidin polymer composition while SP-303 has higher purity, for example 99-100%.

In another embodiment, the powder form of the composition is provided in a container, such as a bag, box, bucket, or pail (e.g., 5 lb. to 25 lb. pails), in which the powder can be in an amount of, for example, 100 grams (g) or more, and can optionally include a measuring device, such as a scoop, cup, spoon, trowel, dipper, or ladle. Such containers encompass, for example, an individual daily dose of the composition; or an amount suitable for multiple treatments, e.g., a two-day treatment, three-day treatment, four day treatment, etc. An effective amount of the powder can also be mixed with feed for consumption by the young animals, e.g., calves, or adult animals in need thereof. Dosages may be 200-800 mg per day.

In an embodiment, the proanthocyanidin polymer composition or prebiotic composition is administered or delivered to a neonatal animal afflicted with diarrhea and in need thereof by providing the compound as a bolus. In an embodiment, the proanthocyanidin polymer composition formulated as bolus. i.e., a pill, capsule, or tablet, is orally administered to the neonatal animals afflicted with diarrhea or symptoms thereof, e.g., calves, foals, lambs and kids, directly in the mouth. In an particular embodiment, the treatment regimen comprises administering a dose of 250 mg of the product, e.g., as embraced by one bolus per sick animal for a determined time period, for example, for one, two, or three or more days. The product can be provided to an animal in need thereof in portions of the complete dose, in which the portions are administered one or two or more times per day. Alternatively, the complete dose can be administered to an animal in need thereof one, two, three, four, or more times per day. In a particular embodiment, the treatment encompasses a dose of 250 mg given two times a day. In a particular embodiment, the treatment encompasses a dose of 2-10 mg/kg given two times a day or given four times a day for three days. In another embodiment, the treatment encompasses an oral bolus dose given two times a day for 3 days, or three times a day for three days, or four times a day for three days. In another embodiment, the treatment encompasses an oral bolus dose of 250 mg given two times a day for 3 days. In an embodiment, the dose is the Croton lechleri proanthocyanidin polymer composition, SB-300, in enteric form or in non-enteric form, e.g., a reconstituted powder form.

In an embodiment, the composition is in a gel or gel formulation. In an embodiment, the gel is contained or preloaded in a delivery device, such as a syringe or other type of injector or delivery system, especially for oral delivery. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. A gel formulation is particularly suited for administration to neonatal and young foals, but also is applicable for other neonatal animals, such as those described herein. In an embodiment, the gel is not contained in a delivery device, but is administered to the roof of the mouth of the animal, particularly one that is too incapacitated or ill to eat or drink, thereby eschewing an oral or other mode of administration. In an embodiment, gels are prepared for oral delivery and contain copolymers, such as poloxamers and Pluronics of different types, e.g., Pluronic F. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein.

In another embodiment, the composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste formulation contains beads with enterically coated SB-300 or SP 303, which is administered to an animal, such as a horse foal, at a dose of 2 mg/kg. More particularly, the paste formulation containing enterically coated SB-300 beads is administered to the foal at a dose of 2 mg/kg, twice a day for three days. More particularly, the paste formulation containing enterically coated SB-300 beads is administered to the foal at a dose of 2 mg/kg, three times a day for three days. More particularly, the paste formulation containing enterically coated SB-300 beads is administered to the foal at a dose of 2 mg/kg, four times a day for three days. In an embodiment, the paste containing enteric protected SB-300 beads is administered twice a day at twelve hour intervals.

In other embodiments, the proanthocyanidin polymer compositions or polyphenol-containing extracts of the invention are formulated in an animal feed composition for administration to a young or adult animal.

The routes of administration of the C. lechleri proanthocyanidin polymer product to afflicted animals are not intended to be limiting. Illustratively, administration can be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals.

Dosage forms can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles, delayed release tablets/pills, dispersible tablets/pills, effervescent tablets/pills, extended release tablets/pills, orally disintegrating tablets/pills, tapes, or troches/lozenges. The dosages can be provided as formulations, compositions, pharmaceutically acceptable formulations and compositions, physiologically acceptable formulations and compositions, including pharmaceutically and physiologically acceptable carrier, excipients, diluents, or vehicles as known and used in the art.

For oral administration, the C. lechleri proanthocyanidin polymer product or prebiotic product, or a composition thereof, is preferably encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating diarrhea, such as secretory or watery diarrhea, and its symptoms in neonatal and young animals or adult animals in accordance with the methods of the invention, a therapeutically acceptable form of the C. lechleri proanthocyanidin polymer composition, including a C. lechleri botanical extract, is administered, particularly orally administered, in an amount ranging from 0.1-100 mg/kg per day, once, twice or more daily. In other embodiments, the amount can range from about 0.1 to about 10 mg/kg/day, once, twice or more daily; or from about 0.1 to about 25 mg/kg/day, once, twice, thrice, four times, or more daily; or from about 0.1 to about 30 mg/kg/day, once, twice or more daily; or from about 0.1 to about 40 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice, thrice, four times, or more daily; or from about 1 to about 5 mg/kg/day, from about 1 to about 8 mg/kg/day, from about 1 to about 10 mg/kg/day, or from about 2 to about 4 mg/kg/day once, twice, three times, four times, or more daily. In an embodiment, the amount of the C. lechleri proanthocyanidin polymer composition for administration is 2 mg/kg two times a day. In another embodiment, the amount of the C. lechleri proanthocyanidin polymer composition for administration is 2 mg/kg three times a day. In another embodiment, the amount of the C. lechleri proanthocyanidin polymer composition for administration is 2 mg/kg four times a day. In an embodiment, the 2 mg/kg dose is administered twice a day for three days. In an embodiment, the 2 mg/kg dose is administered three times a day for three days. In an embodiment, the 2 mg/kg dose is administered four times a day for three days. In a more particular embodiment, SB-300 enteric beads are formulated in a paste which is administered to a neonatal or young animal, e.g. a horse foal, or an adult animal at a dose of 2 mg/kg two times a day for three days, or three times a day for three days, or four times a day for three days, or longer than three days. In other embodiments, the foregoing amounts, and 1-10 mg/kg, or 2-4 mg/kg, of the C. lechleri proanthocyanidin polymer composition are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in the neonatal and young animals.

In other embodiments, for the treatment methods, a suitable dose for the C. lechleri proanthocyanidin polymer product, or the C. lechleri proanthocyanidin polymer composition, such as SP 303 or SB-300, or botanical extract may range from about 1 mg to about 1000 mg, either daily or multiple times per day. In an embodiment, a suitable dose may range from about 10 mg to about 500 mg, either daily or multiple times per day. In an embodiment, a suitable dose may range from about 50 mg to about 350 mg, either daily or multiple times per day. In an embodiment, a suitable dose may range from about 30 mg to about 400 mg, either daily or multiple times per day. In an embodiment, a suitable dose may range from about 100 mg to about 250 mg, either daily or multiple times per day. In an embodiment, a suitable dose may range from about 50 mg to about 300 mg, either daily or multiple times per day. It will be understood that the amount and dose ranges described herein include the lower and higher amounts specified, as well as amounts in between. The doses administered multiple times per day can be given for consecutive days, e.g., two days, three days, four days, five days, six, days, seven days, or more, in some embodiments. A dose administered multiple times per day may embrace two, three, four, five, six, or more times per day. Other dosing schedules, such as every other day, or every third day, every fourth day, etc. are embraced by the invention. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer is SB-300. In an embodiment the C. lechleri proanthocyanidin polymer is enterically coated SB-300. In an embodiment the C. lechleri proanthocyanidin polymer is non-enterically coated SB-300.

In some embodiments, daily doses, including multiple daily doses, e.g., twice or three times a day, of the C. lechleri proanthocyanidin polymer product or prebiotic composition may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or there between) per animal. Administration schedules may also be altered to achieve a therapeutically effective concentration of the C. lechleri proanthocyanidin polymer to treat the diarrhea and its symptoms as described herein. By way of specific, yet nonlimiting example, a suitable dosage amount for use in the methods according to the invention is 250 mg administered once or twice daily. In some embodiments, the compound may be administered once per day, twice per day, thrice per day, 4 times per day, 5 times per day, 7 times per day or 10 times per day. Often the dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

It will be understood that the dose amount actually administered can be determined by the practitioner, in the light of the relevant circumstances, including the severity of the disease, condition, or symptoms thereof being treated, the form of the product to be administered, the age, weight, and response of the individual animal receiving treatment, as well as the chosen route of administration. For all amount or dose ranges, it is intended that the lower and upper amount or dose is included in the range.

The methods of the invention further embrace the administration of pharmaceutically acceptable formulations of the proanthocyanidin polymer composition or prebiotic compositions either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents, although because many of the microorganisms and pathogens that are associated with diarrhea induction in neonatal and young animals concomitantly decrease gut motility, the use of anti-motility drugs may be contraindicated. Natural biological products, e.g., Lactobacillus, Bifidobacterium, Streptococcus faecium or Saccharomyces, or probiotics, may also be employed as additives to restore the natural balance of intestinal flora in the affected neonatal animals.

### EXAMPLES

### Example 1

### Control of Diarrhea in Neonatal Camel Calves Treated with a Composition Containing a Proanthocyanidin Polymer or Oligomer Extract from Croton Lechleri

Neonatal diarrhea remains one of the most common causes of death in young camels. Enterotoxigenic E. coli (ETEC) and rotavirus appear to be the most significant infectious causes of diarrhea during the first week of a newborn camel's life. Salmonella is also a problem in older calves. The pathophysiology includes induction by toxins of the secretion of water in the small intestine with secretory diarrhea as a result. Regardless of the pathogens involved in the disease process, treatment is aimed at preventing and correcting the resulting fluid and electrolyte deficits. Calves can lose 5 to 10% of their body weight in water in one day of scouring. It is therefore crucial to limit water loss.

The goal of this study is to confirm the efficacy of a Croton lechleri proanthocyanidin polymer extract composition (NSF) from Napo Pharmaceuticals Inc. in controlling secretory diarrhea in young camel calves (<6 weeks old) in a preliminary study.

Animals: Thirty (30) young calves aged between (1 and 6 weeks) from both genders with clinical signs of diarrhea are enrolled in the study. They are randomly allocated to a treatment group or a control group. Twenty (20) are treated with NSF and ten (10) serve as controls.

Exclusion/Inclusion Criteria: Only calves less than 6 weeks of age with clinical signs of diarrhea are included in the study. Calves with signs of respiratory disease or arthritis are excluded from the study as well as animals showing signs of diarrhea for more than four (4) days.

Treatment Other Than Test Articles: Electrolytes and fluids either oral or IV are administered at the discretion of the attending veterinarian or caretaker. The quantity and frequency are recorded on the data sheet. Antibiotics and NSAIDS may also be used at the discretion of the veterinarian, if they are the “standard of care” treatment The quantity and frequency of their use are recorded on the data sheet. Intestinal protectants and absorbents such as kaolin, activated attapulgite, activated charcoal should not be used, or the animal should be withdrawn from the study. Gastrointestinal modality modifiers (such as loperamide, hyoscine, atropine, dipryrone) are not used in this study.

Test Articles: Croton lechleri extract (NSF) in a small bolus (pill) is administered to the calves directly in the mouth. Sick animals are treated twice a day for 3 days with a dose of 250 mg of product (one pill) per calf, together with the “standard of care treatment” (2 liters of oral electrolyte twice a day or 4 liters of intravenous fluid per day). Treatment group allocation in chronological order includes: the 2 first animals are treated (from Group 1), the third is a control (from Group 2), 2 treated, one control, and so on. The controls receive the standard of care treatment without the NSF pill. The “standard of care” treatment is the same for both groups.

Clinical Examination: The animals in the study are examined twice a day for 3 days and scored using the parameters detailed below in Table 1.

TABLE 1

| Parameters | | Score |
|---|---|---|
| 1. | Posture/Ability to Stand: | |
| | Animal standing up by itself, alert and active | 1 |
| | Animal standing after encouragement | 2 |
| | Animal standing steadily after lifting | 3 |
| | Animal standing unsteadily | 4 |
| | Animal unable to stand, in sternal or lateral recumbancy | 5 |
| 2. | Degree of Enophthalmos. | |
| | Normal | 1 |
| | Slightly sunken (visible gap between globe and carencula | 2 |
| | lacrimalis but less than 2 mm) | 3 |
| | Severely sunken | |
| 3. | Suckling Reflex | |
| | Strong | 1 |
| | Weak | 2 |
| | Absent | 3 |
| 4. | Hydration by Skin Tenting: | |
| | Pinch a fold of skin on the neck and count the seconds it takes to flatten: | |
| | Two seconds = normal | 1 |
| | 2 to 6 seconds = 8% dehydration | 2 |
| | More than 6 seconds = severe dehydration 10% | 3 |
| 5. | Diarrhea: | |
| | Normal feces, consistence of pudding sample retains | 1 |
| | original shape if placed in a container | |
| | Semi-solid, less firm such as yogurt. Sample spread across the bottom of the | 2 |
| | container but it is not liquid | 3 |
| | Sample is liquid with the consistency of maple syrup | 4 |
| | Consistency of apple juice but some fecal matter still seen. | 5 |
| | Consistency of water, no fecal matter, some mucus or blood could be seen | |
| 6. | Body Temperature: | |
| | Data will be recorded on the data sheet provided. | |

### Example 2

### Evaluation of the Effect of Oral Administration of a Croton Lechleri Proanthocyanidin Polymer Composition on the Fecal Scores of Salmonella Typhimurium-Infected Neonatal Bovine Calves Afflicted with Diarrhea

Diarrhea remains an important cause of morbidity and mortality in neonatal calves (P. Constable, 2004, J Vet Intern Med., 18:8-17). The economic losses associated with this disease are due not only to the resulting mortality, but also to the retarded growth of the animals, the cost of both the veterinary care and the drugs used to treat the infection, and the increased labor involved (D. C. de Graaf et al., 1999a and 1999b, Int J Parasitol., 29:1269-1287 and 1289-1306). Several enteropathogens are associated with diarrhea in neonatal calves, the most prevalent being Escherichia coli, Clostridium perfringens, Salmonella spp., Cryptosporidium spp., and rotavirus and coronavirus, with their relative importance varying by geographic region (D. R. Snodgrass et al., 1986, Veterinary Record, 119:31-34; E. E. Younis et al., 2009, Res Vet Sci., 87:373-379).

A small completely randomized study was conducted to evaluate the effect of oral administration of 250 mg of a Croton lechleri proanthocyanidin polymer composition, i.e., oral SB-300, on fecal consistency of bovine calves infected with Salmonella typhimurium and receiving treatment twice daily for 3 consecutive days. Fecal consistency scores were determined throughout the treatment period. A total of 82 calves were randomly allocated into one of two treatment groups; 39 calves were allocated into the control groups and 43 calves were allocated into the treatment groups. All calves were clinically affected with diarrhea induced by Salmonella infection and received palliative therapy according with the farm standard operating procedures. In addition, calves allocated the treatment group received the same palliative care and were treated orally with the Croton lechleri proanthocyanidin polymer SB-300 composition, as above.

Calves were scored for fecal consistency using a three level score system; 0=solid/normal well-formed feces, 1=pasty feces, and 2=watery diarrhea. Each calf received a total of 6 fecal scorings (twice daily, morning and afternoon) for three days following the diarrhea diagnosis. Data were analyzed using repeated measures ANOVA.

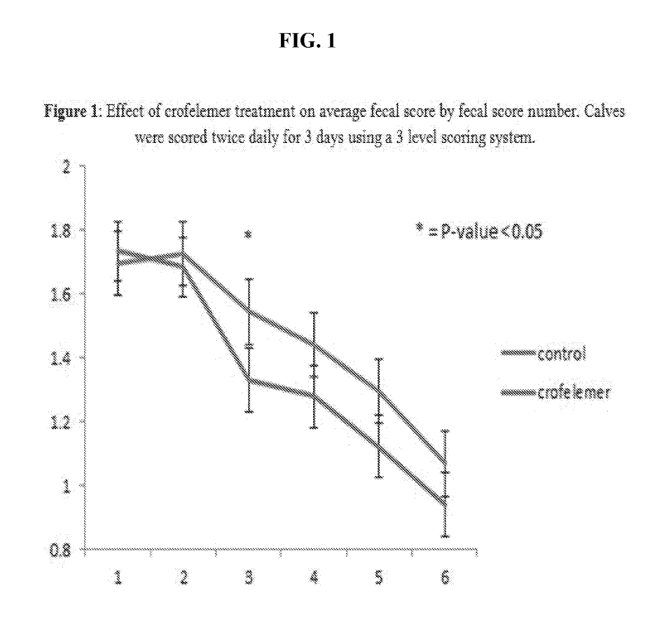

Treatment with the Croton lechleri proanthocyanidin polymer extract composition (SB-300) had a strong tendency to improve (i.e., decrease) fecal scores (P value=0.05). Overall, the average fecal score for control calves was 1.46 and for the treatment calves it was 1.34 (P value=0.05). Fecal consistency scores were similar between treatment group at the beginning of the study. Calves treated with the Croton spp. proanthocyanidin polymer extract composition demonstrated faster improvement on diarrhea scores, starting on the second day of treatment (See, FIG. 1).

Because the commercial farm where the study was conducted was undergoing a severe Salmonella typhimurium outbreak, the calves enrolled in this study were determined infected with Salmonella bacteria, which mainly cause malabsorption diarrhea. Thus, it is possible that the effect of the SB-300 composition on fecal scores of the study calves would be increased if hypersecretory diarrhea were evaluated rather than Salmonella-induced malabsorption-type diarrhea, as the former type of diarrhea is more readily treatable by the C. lechleri proanthocyanidin polymer's typical mechanism of action.

### Example 3

Treatment of E. Coli Challenged Calves with a Croton Lechleri Proanthocyanidin Polymer Extract Composition SB-300

This Example describes another bovine calf study that was conducted in the isolation unit at Cornell University, Ithaca, N.Y., in which the calves were treated with either enteric or non-enteric formulations of crofelemer, the Croton lechleri proanthocyanidin polymer extract composition. All calves were male Holsteins from the same dairy farm in upstate New York. The calves' weights at birth ranged from 57 pounds to 106 pounds.

In this study, calves were clean caught and within two hours were transported to an isolation facility for research animals (Cornell Animal Research Facility, Ithaca, N.Y.). Calves were individually housed in 16 square meter rooms with controlled temperature and humidity. For the clinical trial, the calves were challenged using an enterotoxigenic E. coli serotype O9:K35:K99 (ATCC #31616). After standard bacterial activation, E. coli serotype O9:K35:K99 were grown in Trypticase soy broth (BBL Microbiology Systems) for 8 hours and then on Minca-IsoVitaleX (BBL) agar for 18 hours at 37° C. The bacteria were suspended in phosphate-buffered saline with 10% dimethyl sulfoxide and stored in 10-mL aliquots at −70° C. The mean inoculum titer was 4×10¹⁰ colony-forming units (CFU) per 10 ml (CFU/10 mL).

All calves were challenged at the research facility within 5 hours of life, e.g., 1 to 5 hours after birth. A mixture of freshly-prepared 1 liter (L) of antibiotic-free colostrum replacer plus 10 mL of thawed E. coli inoculum, described above, were administered to the calves via esophageal feeder. Calves were fed non-medicated milk replacer (22-20) on a 10% body weight daily basis, twice a day, e.g., at 0600 h and 1800 h. All calves were kept in the study until 25 days of life with ad libitum access to water. In addition, calf starter (Calf starter 18% CP, DuMOR®, Tractor Supply Co.) was also available ad libitum starting on the seventh day of life.

In this study, 60 calves were enrolled in three groups. Group 1: Twenty (20) calves received 250 mg of the Croton lechleri proanthocyanidin polymer extract composition SB-300 as an enteric coated tablet twice a day for three days. Group 2: Twenty one (21) calves received 250 mg SB-300 as a non-enteric powder reconstituted (dissolved) in milk or oral electrolytes twice a day for three days. Group 3: Nineteen (19) calves received a placebo (enteric coated tablets containing sugar and iron oxide), either as a tablet or a reconstituted powder twice a day for three days, as control.

Treatments were administered twice daily before each meal (a total of six treatments per calf), with the first treatment administered before the first meal (approximately 12 hours after bacterial challenge). Calves were weighed at birth and again at 10, 15 and 25 days of life. Fecal scores (plus 20 grams of fecal sample were collected), skin turgor and eyes recession (indicative of dehydration) were evaluated twice daily for each calf from birth until day 10, at day 15, at day 25.

Calves were treated at the onset of diarrhea. In general, diarrhea was treated according to the dehydration level and attitude of the calf. Calves having diarrhea and slight dehydration, but having normal appetite, were offered oral electrolytes (Re-sorb, Pfizer); calves affected with diarrhea, dehydration, and poor appetite, but still ambulatory, were fed 2 liters of oral electrolyte mix; and calves unable to stand and severely dehydrated were treated with 4 liters of intravenous fluid (Plasma-lyte 148 and 5% dextrose injection; Baxter Corporation).

Calves were closely monitored for dehydration, appetite, attitude, fecal consistency and any adverse health disorder (Table 2). A first blood sample was collected from all study calves within 12 hours post challenge. For every calf, fecal samples were collected twice daily in order to analyze dry mater of the feces, and a daily blood sample collection was performed to monitor precisely the hydration status (total protein, packed cell volume (PCV), chemistry). Daily starter intake and milk intake were recorded for the entire study period. Immunoglobulin-G levels were measured for blood collected 48 hour after birth using an ELISA kit (Bethyl Laboratories, (Montgomery, Tex.). For all daily blood samples; serum total protein, PCV and the acid-base serum status (full blood chemistry) were conducted at the Clinical Pathology laboratory (Cornell University, Ithaca, N.Y.). Calf health-related events used as criteria for clinical diagnosis and assessment of animal health are presented in Table 2 below.

Fecal scores (Table 2) were evaluated as follows: they were based on diarrhea severity and a 5 point scale to assess visually calf diarrhea on milk-fed calves. Fecal scores were categorized as follows: 0=formed feces with normal color; 1=pasty (semiformed) feces with normal color; 2=liquid (watery) feces with normal color; 3=watery feces with normal color (and/or mucous); and 4=watery feces with abnormal color (and/or blood in feces).

TABLE 2

| Health condition | Clinical signs | Score |
|---|---|---|
| Fecal Consistency | Formed | 0 |
| | Semiformed | 1 |
| | Watery | 2 |
| | Watery with mucous | 3 |
| | Blood in feces | 4 |
| Hydration | Normal appearance | 0 |
| | Sunken eyes | 1 |
| | Skin tented 5 to 10 s | 2 |
| | Skin tented ≧ 10 s | 3 |
| Attitude | Alert | 0 |
| | Depressed | 1 |
| | Non responsive | 2 |
| Appetite | Normal | +++ |
| | Consuming < 1/2 bottle | ++ |
| | Consuming < 3/4 bottle | + |
| | (orogastric tube) | |

Preliminary results of this study showed a difference in response between placebo treated calves and those treated with SB-300. In addition, a difference in the response was observed between calves administered the enteric form of SB-300 and those administered the non-enteric, dissolved powder form, as observed before the end of treatment, as presented in FIG. 2. FIG. 2 shows that the difference between the average from the group of calves treated with enteric coated tablets of SB-300 and the average from the group of calves treated with the placebo was statistically significant before the end of treatment. (Day 4 AM, p<0.002, ANOVA). Thus, the calves treated with enteric SB-300 exhibited a significant improvement in fecal consistency over time of treatment.

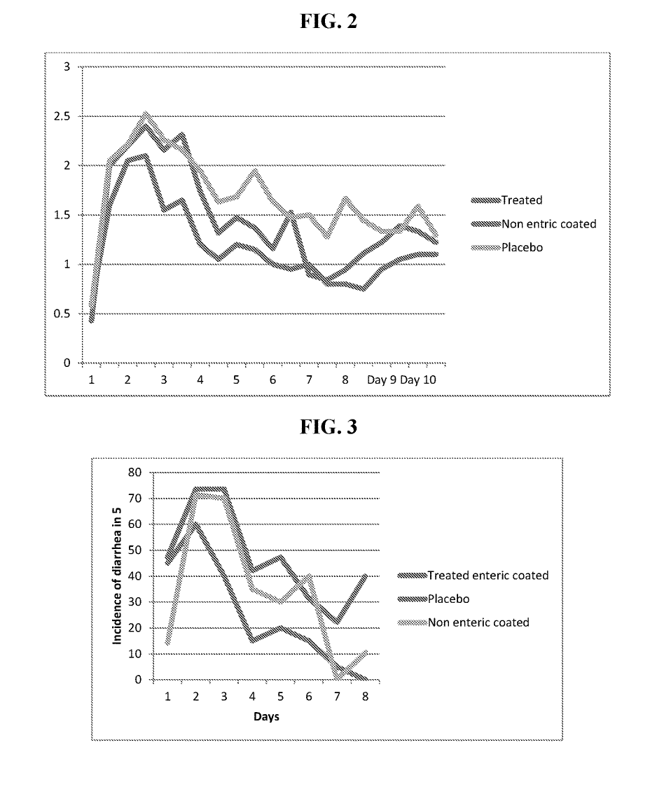

With respect to animal morbidity, the percentage of calves having watery diarrhea (score of 2 and above) are presented in FIG. 3. Each calf was scored twice daily. The difference between the calves in the group treated with the enteric tablet form of SB-300 and the calves in the group treated with placebo is statistically significant on day 4 AM (p<0.05 Fischer test). Thus, the enteric SB-300 treated calves showed a better response (improvement in the diarrhea condition; fewer calves having watery diarrhea) than did those receiving the non-enteric form at the time of evaluation. (See, FIG. 3).

In addition, the calves treated with enteric form of the SB-300 Croton lechleri proanthocyanidin polymer composition showed a greater average weight gain during the 25 day observation period compared with animals treated with a reconstituted powder form of SB-300 or placebo. (Table 3). This was an unexpected finding, especially because the pH in the calf stomach is relatively high, e.g., it can reach ˜pH 6.0 at the time of feeding, compared with a low, acidic pH in the stomachs of other animal species. In view of the high, less acidic pH in the calf stomach when treated with the SB-300 Croton lechleri proanthocyanidin polymer composition at the time of feeding with milk, it was unexpected that an enteric coated formulation would provide a treatment benefit for the calves, or would yield an improvement in the animal's diarrhea condition, relative to a non-enteric coated form of the composition. The finding that an enteric coated Croton lechleri proanthocyanidin polymer composition (SB-300) provided a better response in the young calves following its administration was a surprising and beneficial discovery related to the study.

An analysis of calves monitored during the progression of the study described in this example showed the average weight gain of animals in lbs. between day 1 and day 25, as presented in Table 3. Animal mortality was also monitored during the 25 day study period. Table 3 also shows that ten out of sixty (10/60 ) calves died during the 25 days of the study. Deaths were seen among the groups of calves treated with either SB-300 powder or placebo; only one death occurred in the calf group treated with SB-300 in enteric coated tablet form.

TABLE 3

| | | Mortality (%)/ | Average weight |
|---|---|---|---|
| | Number | (Number of | gain after 25 |
| Treatment | of calves | deaths) | days (lb.) |
| SB-300 enteric | 20 | 1 (5%) | 15.5 lbs. |
| coated tablets | | | (281 g/day) |
| SB-300 powder | 21 | 5 (23.8%) | 11.12 |
| Placebo | 19 | 4 (21%) | 12.1 lbs. |
| | | | (219 g/day) |

A preliminary analysis of the above study results showed the percent mortality of animals treated with placebo to be 21.5%, which is very close to the value determined at completion of the study and presented in Table 3; the number of animal deaths in this group was the same. In the preliminary analysis, the average weight gain after 25 days for animals treated with SB-300 enteric coated tablets was determined to be 11.94 lbs., and for animals treated with placebo, the average weight gain after 25 days was determined to be 9.30 lbs. Thus, the preliminary values and results are highly consistent with those determined at completion of the study, as presented in Table 3. The results from this study demonstrate a clear benefit to the use of SB-300 in enteric form to prevent calf mortality in animals sickened by diarrhea induced by infection with E. coli. In addition, the calves gained more weight when treated with enteric tablets of SB-300. A reduction in mortality and weight gain in animals treated with an SB-300 Croton lechleri proanthocyanidin polymer composition, particularly, the enteric form of SB-300, elucidates the advantages of the treatment methods of the invention. Increased weight of the study animals allowed the animals to become healthier and stronger during their treatment with the SB-300 Croton lechleri proanthocyanidin polymer composition. Decreasing animal morbidity, increasing weight gain and producing healthier animals with a reduced number of treatments all impact the health, nutrition and overall quality of animals in animal-focused industries worldwide. This further translates into commercial, financial and economic advantages and benefits to those in the calf production industry.

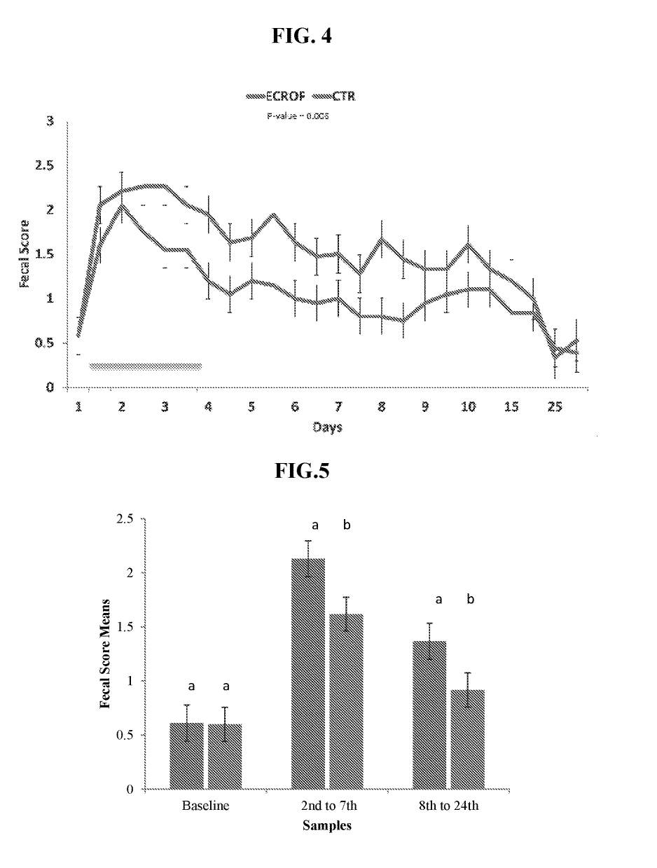

A significant result of the trial described in this Example was observed in connection with the fecal scores and fecal dry matter content of the treated calves of group 1. A multivariate analysis of variance (MANOVA) of the results revealed a significant difference in the fecal scores between the treatment groups during the first 10 days of life. See, e.g., FIG. 4, 20 consecutive samples; p-value=0.018). As can be observed from FIG. 4, calves in the ECROF group had significantly lower fecal scores when compared to calves in the CTR group. As observed from FIG. 5, pre-challenged fecal scores were not significantly different between treatment groups; 0.61 (SEM 0.16), 0.6 (0.15) for CTR, ECROF, respectively (P-value=0.90). Calves in the ECROF group (1.61±0.15) had significantly lower fecal scores during treatment when compared to control group (2.13±0.16, p-value=0.018). After treatment cessation, fecal scores were significantly lower for calves in the ECROF group (P-value=0.012) when compared to control calves.

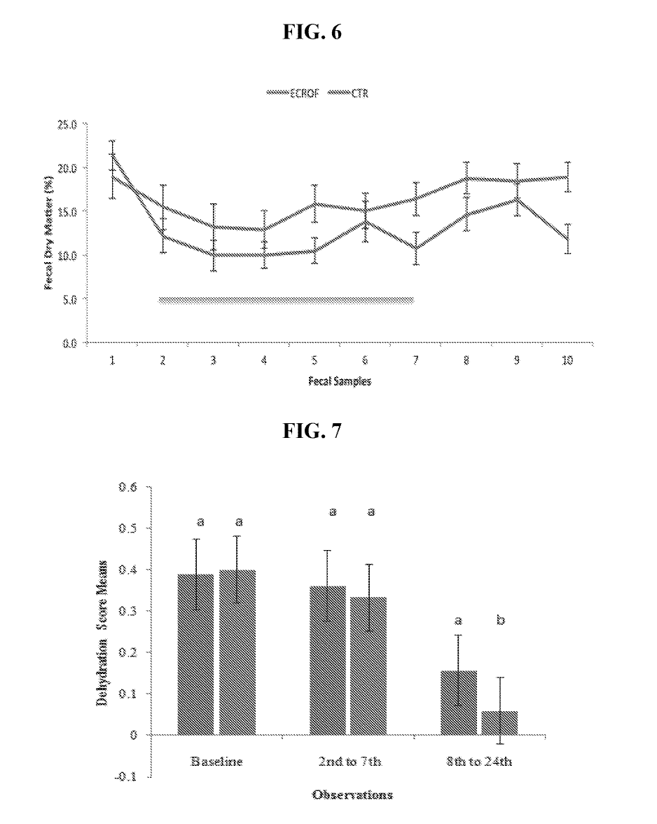

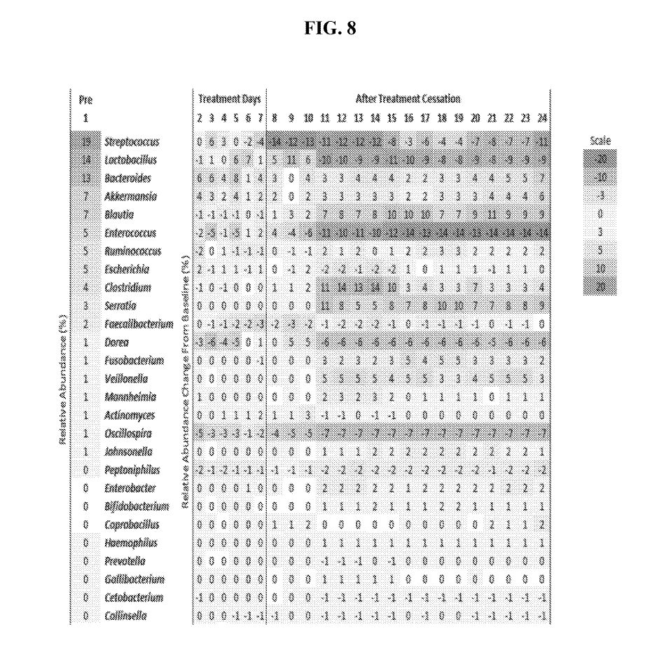

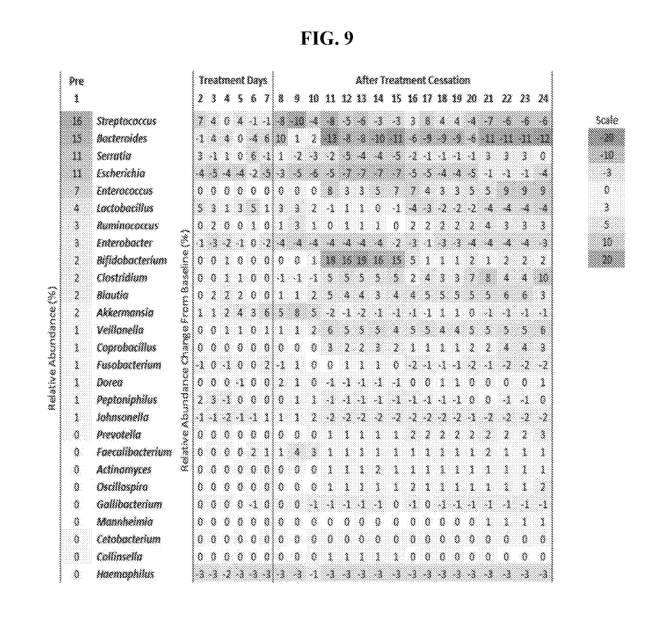

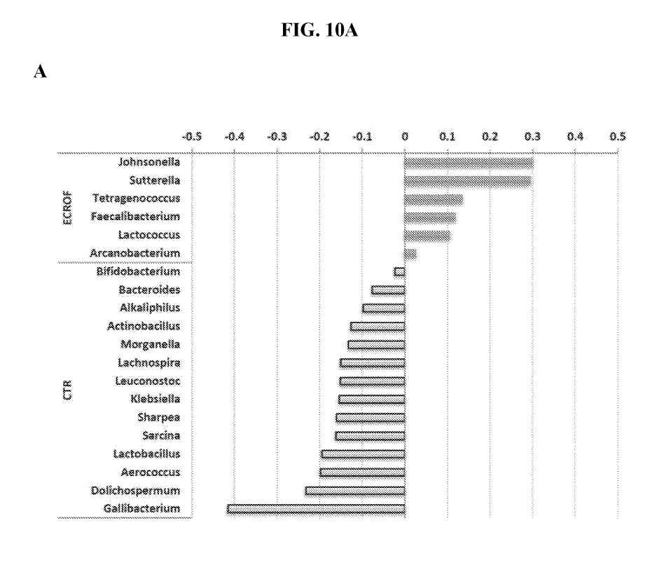

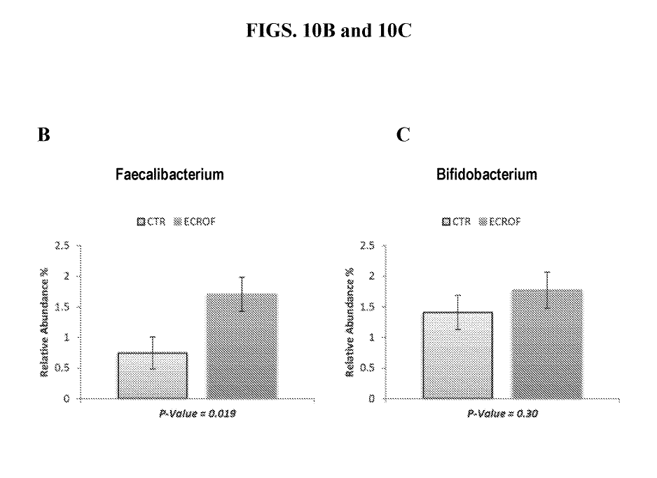

The fecal scoring data of FIGS. 4 and 5 were confirmed by the data reflecting the dry matter in the feces (See, e.g., FIG. 6). In FIG. 6, calves in ECROF group had significantly higher fecal dry matter content when compared to calves in CTR (p-value=0.03).

In addition, no difference was observed in dehydration score between treatment groups before challenge (P-value=0.80) nor during treatment days (FIG. 7, p-value=0.67). However, after treatment cessation, lower dehydration scores were observed for the calf group treated with ECROF (P-value=0.03) compared to the dehydration scores in the control group.

A surprising and unexpected discovery resulting from this trial is that the effect of the administration of the C lechleri proanthocyandin extract containing product on diarrhea and dehydration lasted much longer than the period of time the product was given to the calves. While it was previously believed that the active component of the Croton lechleri derived product does not have a carryover effect, the results obtained from the clinical study and presented here surprisingly demonstrate that early administration of the product may induce beneficial changes in the intestines of the ECROF-treated animals that outlived the course of therapy. For example, administration of the product could favor the development of a healthier GI tract microbiota.

### Example 4

### Treatment of Diarrheic Calves with a Croton Lechleri Proanthocyanidin Polymer Extract Composition SB-300—Field Study

A field trial study was conducted on 200 newborn Holstein heifer calves as study animals. An object of the study was to examine the relationship between treatment of calves with a Croton lechleri proanthocyanidin polymer extract composition, e.g., SB-300, and changes that the treatment leads to in preweaned weight gain and daily fecal dry weight in treated animals.

The calves were randomly enrolled into two treatment groups at the onset of diarrhea. One group of 100 calves received placebo. The other group of 100 calves received the Croton lechleri proanthocyanidin polymer extract composition (such as Neonorm™) in an amount of 250 mg. The calves were treated at the onset of diarrhea, twice daily for three days. Data were collected on: (i) weight—at birth, at onset of diarrhea, at end of treatment, and at weaning; (ii) fecal scores and samples—collected at onset and after each treatment; and (iii) mortality and morbidity—until day 45 of life.

Preliminary results of this study showed that calves treated with the Croton lechleri proanthocyanidin polymer composition showed a strong statistical tendency toward a higher average daily weight gain at 60 days of life (723.3 grams/day) versus placebo treated calves (703.4 grams/day). P-value=0.07. No significant effect of treatment was observed in each treatment group for the parameters of birth weight, weight at treatment, mortality, birth in maternity pen, dystocia, morbidity, (otitis and respiratory disease), age at weaning and initial fecal dry matter. The results showed that a Croton lechleri proanthocyanidin polymer composition or botanical extract, such as SB-300, according to the invention offers a beneficial effect in supporting weight gain in preweaned calves at 60 days of life.

### Example 5

### Treatment of Pre-Weaned Equine Foals Having Watery Diarrhea with a Paste Formulation of SB-300

This example describes a randomized, blind controlled pilot study to assess the safety and efficacy of a paste formulation of SB-300 in pre-weaned foals with watery diarrhea. The study was an exploratory study conducted according to Good Scientific Practices. An aim of the study was to determine the safety, tolerability, and efficacy of a paste formulation of SB-300 on pre-weaned foals when administered orally twice daily for six treatments (BID Group), or orally four times daily (QID Group) for twelve treatments at 2-4 mg/kg/dose. The length of time for the study from screening to final observation and sample collection was 5 to 6 days.

### Background and Rationale

Diarrhea in young equines is very common and there are many causative agents and conditions (viral, bacterial, protozoa, parasites, drug or dietary associated, toxins and changes in the intestinal flora) that manifest with clinical signs of watery diarrhea in these foals. These agents trigger the pathophysiological mechanisms of secretory diarrhea resulting in abnormal ion transport in intestinal epithelial cells. The presence of these abnormal mediators results in changes in intracellular cyclic adenosine monophosphate (cAMP), cyclic guanosine monophosphate (cGMP), calcium and/or protein kinases which, in turn, cause an increase in chloride secretion. Water follows the chloride ions. This increase in intestinal water secretion overwhelms the absorptive capacity of the bowel with resultant diarrhea. Specific treatments aimed at causative agents include the administration of antimicrobials and anthelminthics; while supportive care may include fluids and anti-inflammatories. Symptomatic treatments include bismuth subsalicylate, probiotics and dietary modifications with the principal goal of all therapies being to restore and maintain fluid and electrolyte balance.

There are presently no commercially available products with anti-secretory properties to normalize the intestinal secretion of water in young equines. SB-300 is a purified botanical extract of Croton lechleri and contains ingredients with strong anti-secretory properties and a unique mode of action through modulation and regulation of both cyclic adenosine monophosphate (cAMP)-stimulated cystic fibrosis transmembrane conductance regulator (CFTR) and calcium-stimulated (CaCC) chloride intestinal channels. The compound acts locally in the lumen of the small intestine. The compound is not systemically absorbed, and this is a key factor contributing to the safety of the product. SB-300 has no effect on gut motility and there are no significant drug-drug interactions identified to date.

Probiotics, i.e., live organisms, that when administered at adequate concentrations provide a beneficial effect beyond that of their nutritional value, are used widely in the treatment of diarrhea in humans, small animals and equines. The dosing regimen for probiotic use and probiotic brand for diarrhea treatment are highly variable. In some embodiments, the C. lechleri proanthocyanidin product, e.g., crofelemer or SB-300, includes a probiotic, e.g., DIGESTIVE™. The probiotic may be added to the C. lechleri proanthocyanidin product to provide a standard method of care for treating diarrhea.

### Study Conduct

Potential study subjects underwent screening assessments by the Study Veterinarian at each site for determination of eligibility for enrollment into the study. If the pre-weaned foal was eligible for enrollment, he/she was randomized according to the randomization strata assigned. The foal was entered into the Treatment Period, followed by a post treatment Observation Period. From the onset of the Treatment Period to the end of the Observation Period, the Study Veterinarian, or designee, conducted and recorded on the case report forms (CRFs), ongoing fecal scoring, clinical assessments, such as Adverse Events and Concomitant Medications. The total time on study for each foal was 5 to 6 days.

The time of the first dose was recorded in the subject's study CRF as Time 0 (t=0) (start of the Treatment Period up to 72 hours post t=0 timepoint) followed by the Observation Period >72 hours post t=0 and end at hour 144 post t=0 timepoint. Dosing and assessment times were recorded on the CRFs by the Study Veterinarians and/or designated site staff. Fecal and blood samples were collected and analyzed at baseline and at the end of the Treatment Period along with the ultrasound assessment in the observation period. Abdominal ultrasounds were conducted at baseline, up to twelve hours upon completion of both the Treatment Period and Observation Period. Housing of foals and mares were as per study site standards of care and were maintained throughout the Treatment and Observation Periods. Feed was provided in an amount and manner that afforded the nutrient and energy requirements to ensure the health and well-being of the study subjects. Foal feeding schedules were maintained pursuant to their usual frequency. Mare feed was given once or twice daily or ad libitum. Water was also available ad libitum.

### Trial Design

The trial was a multi-site randomized, blind-controlled exploratory, safety, and efficacy study in pre weaned foals between birth and 16 weeks of age. Approximately 80 pre-weaned foals were enrolled to ensure at least 60 evaluable foals. The study consisted of 3 treatment groups. Twenty foals were enrolled in each treatment group. Enrolled foals received 1) active investigational veterinary product (IVP), such as SB-300, 4 times daily, 2) active IVP twice daily and placebo twice daily, or 3) placebo 4 times daily. The syringe contained IVP or placebo. The veterinarian at the study site identified potential candidates for the trial from the current patient population and potential study subjects underwent baseline assessments for eligibility evaluation. Upon eligibility determination based on the Inclusion/Exclusion Criteria, the foal was randomized beginning with the lowest number in the assigned Treatment Group, followed by sequential numbering of the next treatment option available to the site. The site veterinarian and staff were instructed on the protocol requirements, including administration of the investigational product (IVP) and the required study procedures and assessments. The abdominal ultrasounds were conducted by a veterinarian assigned by the sponsor veterinarian

Approximately 80 pre-weaned foals were randomized to 3 different treatment groups to ensure the completion of 60 evaluable cases for the statistical analysis. Pre weaned foals with watery diarrhea (fecal score of 3 or 4) from birth to 16 weeks of age comprised the study population with an equal number of these foals distributed across each treatment and placebo group. Animal characteristics and inclusion criteria for the study animals are presented in Table 4 below.

TABLE 4

| | |
|---|---|
| Species: | Pre-weaned foals |
| Breed: | All |
| Source: | Client-owned |
| Number: | ~80 |
| Gender: | Males or Females |
| Weight Range: | 35-200 kg |
| Age Range: | Birth-16 weeks of age |
| Physiological Status: | Pre-weaned foals with a |
| | screening fecal score of 3 or 4 |

In this randomized, blinded, placebo-controlled study, there were two test materials: one contained the investigational veterinary product, SB-300 (IVP) and a probiotic (Digestiv™); the second was a placebo. Both test materials were formulated as a paste and packaged in identical 30 ml metered syringes. Both test materials contained the same formulants. All formulation components are considered safe for use with animals. The distinction between IVP and placebo syringe was blinded to the person administering the dose. There were three arms in the study. Each arm utilized the same number of syringes per foal. Each syringe was identified with a unique alpha-numeric code that connected it with one of the three arms in the study. Syringes were prepared as test kits; one test kit per foal enrolled in the study. Test kits were assembled at the manufacturing facility under QC supervision and shipped to the study site with a unique number that associated it with one of three arms.

The non-placebo syringes contained 400 mg of crofelemer. The foals of least weight in the study (e.g., 35 kg) received a 10 mg/kg dose. The heaviest foals in the study (e.g., 200 kg) received a 2 mg/kg dose. This dose range (2-10 mg/kg) was justified based on a safety study run in foals at 5× the typical dose of 2-4 mg/kg. All foals enrolled in the study regardless of weight were administered the entire contents of a 30 mL syringe at each dosing time point.

### Screening and Treatment Periods

During the screening period, a medical history, a complete physical exam and an initial fecal collection for analysis were conducted along with blood sample collection to establish baseline parameters for each foal. Additional fecal samples and rectal swabs were collected and stored. An initial ultrasound examination of the abdomen was performed prior to administration of the first dose to assess bowel wall thickness and consistency of intestinal contents. Any findings during the Baseline Period, prior to treatment administration, were noted as Medical History and documented on the Medical History Form. As each foal was deemed eligible to participate in the study, blinded randomization into either placebo QID, treatment BID with placebo BID, or treatment QID groups was conducted. A study site completed the Screening Log for any foal screened for the study regardless of whether the foal was subsequently randomized into the Treatment Period or was Screen failed. A screening log was provided to each site.

Upon eligibility determination, the Treatment Period began at the time of first dose (t=0) and continued to 72 hours (+/−2) after the initial dose. Each foal was randomized to 1 of 3 treatment groups. An abbreviated dosing and activity schedule is presented in Table 5 below.

TABLE 5

| | Treatment Period | Observation Period |
|---|---|---|
| Screening | 0 to 72 Hrs | >72 Hrs to 144 Hrs |
| Medical History/PE | Physical Exam | Physical Exam |
| HEME/CHEM | | HEME/CHEM |
| Body Weight | | Body Weight |
| Fecal Analysis & | Fecal Culture | |
| Culture | (T₁₂ & T₄₈) | |
| Ultrasound | Ultrasound | Ultrasound |
| Group Allocation | Randomization/Dose | |
| | Administration | |
| Fecal Scoring | At least 4 Times Daily | At least Twice Daily |
| Fecal Dry Weight | Fecal Dry Weight | Fecal Dry Weight |
| | Twice Daily | Twice Daily |
| Fecal Swab | 1 x Daily | 1 x Daily |

Fecal collection for analysis was conducted at baseline only, except for Salmonella analysis, which was conducted at baseline, 24 and 48 hours (+/−6) hours post t=0. Feces (10 grams) were collected from all groups and sent for analysis. Rotavirus antigen test and Clostridial toxin tests were conducted along with fecal culture analysis to include screening for pathogenic E. coli, Salmonella, Shigella and other aerobic pathogens. Sampling for fecal dry weight was collected and stored at baseline and twice daily during the Treatment and Observation Periods described below. No fecal dry weight analysis was intended for this study.

Fecal scoring was conducted during each 6 hour (h) period starting 0-6 h, >6-12 h, >12-18 h, >18-24 h, >24-30 h, >30-36 h, >36-42 h, >42-48 h, >48-54 h, >54-60 h, >60-66 h, >66-72 h. At each evaluation, the number of stools was recorded, scored, and documented on the CRF. After each evaluation, the stalls were cleared of any fecal material. Attempts were made to collect additional fecal samples twice daily for fecal dry weight, in addition to collecting a rectal swab sample once daily. Each of these samples was processed and stored. Fecal cultures for detection of Salmonella Spp were performed at Screening and repeated 24 and 48 (+/−6) hours post t=0. An exit ultrasound examination was performed within 12 hours of the last treatment. During the Treatment Period, any newly added Concomitant Medications and observed AEs were recorded as appropriate on the respective Case Report Forms (CRFs).

An Observation Period began upon completion of the Treatment Period (>72 post t=0) and continued until hour 144 post t=0 (+/−2 hours). During this period, fecal scoring was conducted twice daily at >72-84 h, >84-96 h, >96-108 h, >108-120 h, >120-132 h, >132-144 h. Each score was documented on the appropriate CRFs for each foal. The absence of stool at the scheduled assessment time was also noted on the CRFs. During the Observation Period Fecal Dry Weight was assessed twice daily and a Fecal Swap was taken once daily. At the end of the Observation Period (e.g., Hour 144), a complete physical and ultrasound were conducted along with blood sample collection. Upon completion of these tasks, the foal was discharged from the study. Any changes to the baseline laboratory values, or clinical assessments were assessed for Clinical Significance by the site Study Veterinarian and if appropriate, were noted on the Adverse Events CRF.

As the study was designed to determine safety and efficacy of SB-300 in pre weaned foals with diarrhea, outcome evaluations were as follows: 1) changes in stool consistency from baseline through the Observation Period; 2) comparison of clinical assessments, plasma chemistry, and hematology at baseline and post-treatment; 3) comparison of clinical assessments, plasma chemistry and hematology between control and treated groups; and 4) comparison of ultrasound findings at baseline and post-treatment.

### Schedule of Activities

The below table 6 summarizes the schedule of activities in the study.

TABLE 6

| | | | | | Observation |
|---|---|---|---|---|---|
| | | | | | Period⁷ |
| | Screening | Treatment Period | | | >72-144 |
| | Period¹ | 0-72 hours | | | hours |
| Medical History² and | X | | | X | X |
| Physical Exam | | | | | |
| Hematology, Biochemistry | X | | | X | X |
| Body Weight³ | X | | | | X |
| Fecal Collection for | X | X | X | | |
| Analysis⁴ | | | | | |
| Abdominal Ultrasound⁵ | X | | | X | X |
| Randomization | | X | | | |
| Treatment Administered 4 | | X | X | X | |
| times/day according to | | | | | |
| group allocation⁶ | | | | | |
| Fecal Scoring⁸ | X | X | X | X | X |
| Fecal Dry Weight and | X | X | X | X | X |
| Swab⁹ | | | | | |
| Concomitant Medications | X | X | X | X | X |
| Adverse Events¹⁰ | | X | X | X | X |

¹The treatment period may begin immediately after the completion of the screening procedures while the laboratory results are pending.
²Medical History is only conducted at Screening. A Physical Exam is conducted at Screening and at the end of the Treatment and Observation Period. All laboratory and physical exam findings during the Screening Period are captured as Medical History.
³Body Weight is done at Screening to determine group allocation.
⁴Fecal analysis is conducted at screening. Culture for Salmonella spp is performed at Screening and 24 & 48 hours post t = 0.
⁵Abdominal ultrasounds are conducted at baseline and within 12 hours post the last dose given during the Treatment Period and at the end of the Observation Period.
⁶The first dose administered is noted as t = 0. The last dose is administered 72 hours (+/− 2 hours) after the t = 0 timepoint. Each completed foal receives 12 doses respective of their group allocation.
⁷The Observation Period begins >72 hours and ends 144 hours post t = 0.
⁸Fecal scoring is conducted at screening to determine eligibility and continues at least 4 times/day during the Treatment Period plus 24 hours post last treatment and at least twice daily thereafter until the end of the Observation Period. The stalls are cleaned after each assessment.
⁹Fecal Dry weight sampling is conducted at baseline, twice daily during the Treatment and Observation Periods. Swab sampling is conducted at baseline and once daily during the Treatment and Observation Period.
¹⁰Any changes noted after the initial treatment dose and during the Treatment and Observation period are captured as Adverse Events per the Study Veterinarian's discretion.

### Fecal Scoring and Abdominal Ultrasound

A baseline fecal score was determined using the scoring guide below. For eligibility, the foal must have had a score of 3 or 4. Fecal scoring was conducted during each 6 hour period starting 0-6 h, >6-12 h, >12-18 h, >18-24 h, >24-30 h, >30-36 h, >36-42 h, >42-48 h, >48-54 h, >54-60 h, >60-66 h, >66-72 h, >60-66 h, >66-72 h during the treatment period and in conjunction with the dosing times (with a minimum of 4 hours in between assessments), followed by at least twice daily >72-84 h, >84-96 h, >96-108 h, >108-120 h, >120-132 h, >132-144 hthereafter until the end of the Observation Period. Fecal characteristics related to fecal score are presented in Table 7.

TABLE 7

| Fecal Description | Score |
|---|---|
| Well-formed feces | 1 |
| Soft or very soft, moist (cow patty consistency) | 2 |
| Watery, liquid stools with some particulate | 3 |
| matter either evident adhered to the | |
| tail or perineum or upon the surface of the bedding | |
| Severe watery diarrhea with no particulate matter | 4 |
| visible OR no diarrhea seen | |
| but watery staining of the tail, perineum or walls evident. | |
| Hemorrhagic diarrhea | 5 |

An abdominal ultrasound was conducted by the sponsor veterinarian at baseline, up to 12 hours at the end of the Treatment Period and again at the end of the Observation Period. Table 8 presents characteristics of the abdomen for determining an ultrasound score.

TABLE 8

| Ultrasound Description | Score |
|---|---|
| Normal-large colon contents cannot be visualized. | 0 |
| Mild Increase in thickness of abdominal wall but | 1 |
| large colon contents are not visible. | |
| Mild thickening and edema of the bowel wall | 2 |
| with intestinal contents visible | |
| Moderate thickening and edema of the bowel wall | 3 |
| with hypoechoic to hyperechoic intestinal contents visible. | |
| Marked thickening and edema of bowel wall with | 4 |
| anechoic or hypoechoic intestinal contents visible, OR gas echoes | |
| Hyperechoic gas echoes evident within the intestinal wall | 5 |

### Exploratory Outcome Evaluations Utilizing the Abdominal Ultrasound

Additional exploratory outcome evaluations were performed utilizing the abdominal ultrasound and conducting assessments pre- and post-treatment. Evaluations of findings was conducted for each foal pre- and post-treatment and between treatment groups.

Disorders of the abdominal organs represent a large proportion of diseases encountered in equine neonatal medicine. Ultrasound is non-invasive and well tolerated by the foal, and due to the animal's small size and proximity of many abdominal organs, it is an ideal candidate for ultrasonographic evaluation of the viscera.

Small intestinal disorders such as enteritis, duodenitis and large intestinal disorders such as colitis are frequent causes of diarrhea in young foals. Ultrasonographic findings of abdominal disorders are poorly described in foals as compared to adults but common findings include distended, fluid filled bowel with variations in motility patterns. The sonographic appearance of the intestinal fluid may vary from anechoic representing little particulate matter to hyperechoic with a large concentration of particulate matter. Motility disorders may range from ileus (a lack of motility) to hypermotility.

While ultrasound is used clinically to assess the progression or improvement in the disease process, the sequential changes have not to date been well documented. Ultrasound assessment may be used to evaluate the consistency of intestinal contents and changes in bowel wall thickening/edema as indicators of improvement in the absence of fecal production. The rationale for using abdominal ultrasound in this study was to simultaneously evaluate clinical and clinicopathological parameters and correlate them to changes in ultrasound findings with a view to using these findings as exploratory endpoints in this study and endpoints in future studies.

In the present study, prohibited medications included additional Probiotics, oral electrolytes (IV fluids allowed), Oral anti-diarrheal treatments (within 7 days of the first dose administration and while on study). Treatment with rescue medications, per the investigator's discretion, was allowed during the study. Rescue medications administered and indication for use during the treatment period were recorded. If a rescue medication was needed for a suspected adverse event, the event was recorded on the Adverse Event (AE) Form. Adverse events were summarized for each term by treatment group. Additionally, adverse events were summarized by severity and relationship to study drug. For possible differences between treatment groups, continuous safety parameters were analyzed by ANOVA. Within treatment groups AEs were evaluated by paired t-tests or Wilcoxon signed rank tests as appropriate.

### Safety Monitoring

General health observations were conducted daily as per the protocol requirements. However, any observations at any point in time during the course of the study were assessed and medically treated if necessary. If a horse died, it was necropsied. If the horse was moribund, all efforts to euthanize humanely were ensured. The Study Veterinarian or a qualified designee examined the foals for evidence of Adverse Events (AEs). An AE was any observation in the treated animal that was unfavorable and unintended and occurred during or after the use of an investigational veterinary product (IVP), whether or not considered to be the product. AEs may consist of worsening of an existing illness, a newly appearing disease, an accident, a new finding in a clinical laboratory assessment, or a physiological finding during a physical examination. Any AEs (any changes from baseline) noted at the discretion of the Study Veterinarian during the course of the study were reported on the Adverse Event Case Report Form. All AEs that occurred during the study were treated appropriately to protect and ensure the foal's well-being. For an event to be considered an AE, it must have occurred during or after the foal's first exposure to IVP. A Study Veterinarian or a qualified designee was responsible for determining whether or not an AE was severe enough to require the foal's removal from treatment. If this occurred, the foal received appropriate medical care, and the Study Veterinarian ensured the final protocol-specified visit and assessments were conducted. All AEs, serious or not, that resulted in permanent withdrawal from study treatment were immediately reported.

### Body Scoring Guide

The following Table 9 presents a body scoring guide for use in the study.

TABLE 9
* Henneke Body Condition Scoring System describes the condition of each horse on the 1-9 scale:

| Score | Condition | Description |
|---|---|---|
| 1 | Poor | Horse is extremely emaciated. Backbone, ribs, tailhead and hipbones are prominent. |
| | | Bone structure of withers, shoulders and neck are noticeable. No fatty tissue can be |
| | | felt. |
| 2 | Very Thin | Horse is emaciated with slight fat covering over vertebrae. Backbone, ribs, tailhead |
| | | and hipbones are prominent. Withers, shoulders and neck structures faintly |
| | | discernible. |
| 3 | Thin | Fat built up about halfway on vertebrae. Slight fat layer can be felt over ribs, but ribs |
| | | easily discernable. Tailhead is evident, but individual vertebrae cannot be seen. |
| | | Hipbones cannot be seen, but withers, shoulder and neck accentuated. |
| 4 | Moderately | Negative crease along back. Fait outline of ribs can be seen. Fat can be felt along |
| | Thin | tailhead. Hip bones cannot be seen. Withers, neck and shoulders not obviously thin. |
| 5 | Moderate | Back is level. Ribs can be felt, but not easily seen. Fat around tailhead beginning to |
| | | feel spongy. Withers are rounded and shoulders and neck blend smoothly into body. |
| 6 | Moderately | Horse may have slight crease down the back. Soft fat can be felt on the tailhead. Fat |
| | Fleshy | over ribs is spongy. Slight fat deposit can be felt along the sides of the withers, |
| | | behind the shoulder and along the neck. |
| 7 | Fleshy | A crease is seen down the back. Individual ribs can be felt, but noticeable filling |
| | | between ribs with fat. Fat around tailhead is soft. Noticeable fat is deposited along |
| | | the withers, behind the shoulders and along the neck. |
| 8 | Fat | Crease down back is prominent. Ribs are difficult to feel due to fat between them. |
| | | Fat around tailhead very soft. Area along withers filled with fat. Area behind |
| | | shoulders filled in flush with barrel of body. Noticeable thickening of neck. Fat |
| | | deposited along the inner buttocks. |
| 9 | Extremely | Obvious crease down back. Fat can be felt in patches over rib area with bulging fat |
| | Fat | over tailhead, withers, neck and behind shoulders. Fat long inner buttocks may rub |
| | | together. Flank is filled in flush with barrel of body. |

### Outcome Parameters

The following outcome parameters were evaluated: Stool Consistency (changes from baseline over time; a clinical responder was any animal who developed formed stool or had no stool (Fecal Score of ≦3), and maintained formed stool or no stool (i.e., no Fecal Score of 4, 5 or 6) for a minimum of 16 consecutive hours within a 24 hour time period during the 72-hour Treatment Period (T₀ hr-T₇₂ hr); resolution of diarrhea was defined as a Fecal Score of ≦3 (formed stool) at any post-baseline assessment; Time to Last Unformed Stool (TLUS); Ultrasound (changes from baseline to end of treatment period and end of observation period).

### Analysis of Outcome Parameters

Descriptive statistics (number of subjects, mean, standard deviation, minimum, median and maximum values) were presented for continuous variables by treatment group and time point. For categorical parameters, the number and percentage per category were presented for each treatment group. Changes from baseline were evaluated by repeated measures analysis of variance (ANOVA). The models included fixed terms for treatment group, time point, treatment group by time point interaction with site, and treatment group by site interaction as random effects. The baseline value was included in the model as a covariate. The mean value for each 24 hour treatment block was plotted by treatment group.

For the responder analysis, the difference between the number of formed stool scores (1 or 2) and the number of unformed stool scores (3, 4 or 5) was calculated for each animal during the treatment period. In order to assess possible differences between treatment groups, ANOVA modeling with treatment group as a fixed effect and site and treatment by site interaction as random effects was employed. Time to Last Unformed Stool was reported using Kaplan-Meier analysis. The time to last unformed stool was calculated from the date of the first dose to the last time of an unformed stool score (3, 4 or 5). Subjects not achieving a formed stool score of 1 or 2 were censored to the time of the last evaluation. The log-rank test was utilized to compare treatment groups. The 25^{th} quartile, mean time, median time and 75^{th} quartile was presented, if available. All analyses were conducted at a significance level of alpha=0.05, 2-sided, unless otherwise stated. No adjustment was applied for multiple comparisons. Comparisons included each IVP group to placebo and IVP:BID to IVP:QID.

### Study Results

Fecal scores and resolution of diarrhea in foals of the treatment groups, i.e., the BID and QID treatment groups, were analyzed at end of study. As noted above, pairwise comparisons were derived by repeated measures ANCOVA with treatment site, time point, and treatment by time point interaction as fixed effects. The results of the analyses of the treatment groups are presented in the tables below. Table 10 presents Fecal Score Responder Analysis results for foals treated BID with the investigative active SB-300 versus foals treated with placebo in the 0-72 hour and the 0-96 hour time periods of the study. As described, the 0-72 hour study time period is the treatment period; the 0-96 hour study time period is the treatment period plus a 24 hour observation period. Animals whose scores (fecal scores) were 1 or 2 at the times of analysis are considered Responders.

TABLE 10
Fecal Score: Responder Analysis (Active BID)
Responder (Score of 1 or 2)

| Time | | | |
|---|---|---|---|
| Period | | Active BID | Placebo |
| 0-72 h | Responder | 13/19 | 6/17 (35.3%) |
| | (Score of 1 or 2) | (68.4%) | |
| | Pairwise Comparisons* | | 0.0320^{†} |
| 0-96 h | Responder | 15/19 | 8/17 (47.1%) |
| | (Score of 1 or 2) | (78.9%) | |
| | Pairwise Comparisons* | | 0.0328^{†} |

*Pairwise comparisons derived by repeated measures ANCOVA with treatment, site, time point and treatment by time point interaction as fixed effects.
^{†}P-value derived by a generalized linear mixed model assuming a binomial distribution and a logit link

Table 11 presents Resolution of Diarrhea results for foals treated BID with the investigative active SB-300 versus foals treated with placebo in the 0-72 hour and the 0-96 hour time periods of the study. As described for Table 10, the 0-72 hour study time period is the treatment period; the 0-96 hour study time period is the treatment period plus a 24 hour observation period. Animals having scores of 1 or 2 at the times of analysis were considered to be responders with resolution of diarrhea.

TABLE 11
Resolution of Diarrhea: Active BID versus Placebo
Responder (Score of 1 or 2)

| Time Period | | Active BID | Placebo |
|---|---|---|---|
| 0-72 h | Resolution | 14/19 | 7/17 (41.2%) |
| | (Score of 1 or 2) | (73.7%) | |
| | Pairwise Comparisons* | | 0.0895^{††} |
| 0-96 h | Resolution | 16/19 | 9/17 (52.9%) |
| | (Score of 1 or 2) | (84.2%) | |
| | Pairwise Comparisons* | | 0.0704^{††} |

^{††}P-value derived by Fisher's Exact (2-tail) test

Table 12 presents Fecal Score and Responder Analysis results for foals treated QID with the investigative active SB-300 versus foals treated with placebo in a 0-120 hour study time period. The 0-120 hour study time period is the treatment period plus a 48 hour observation period. Animals whose scores were 1 or 2 at the times of analysis are considered Responders.

TABLE 12
Fecal Score: Responder Analysis (QID)
Responder (Score of 1 or 2)

| Time | | | |
|---|---|---|---|
| Period | | Active QID | Placebo |
| 0-120 h | Responder | 15/16 | 6/13 (46.2%) |
| | (Score of 1 or 2) | (93.8%) | |
| | Pairwise | | 0.0199^{†} |
| | Comparisons* | | |

*Pairwise comparisons derived by repeated measures ANCOVA with treatment, site, time point and treatment by time point interaction as fixed effects.
^{†}P-value derived by a generalized linear mixed model assuming a binomial distribution and a logit link

Table 13 presents Resolution of Diarrhea results for foals treated QID with the investigative active SB-300 versus foals treated with placebo in the 0-120 hour study time period. As described for Table 12, the 0-120 hour study time period is the treatment period plus a 48 hour observation period. Animals whose scores were 1 or 2 at the times of analysis were considered responders with resolution of diarrhea.

TABLE 13
Resolution of Diarrhea: Active QID versus Placebo QID
Responder (Score of 1 or 2)*

| Time | | Active | | |
|---|---|---|---|---|
| Period | | QID | Placebo | p-value^{††} |
| 0-120 h | Resolution | 15/16 | 8/13 | 0.0638 |
| | (Score of 1 or 2) | (93.8%) | (61.5%) | |

*Only the sickest animals entering with a fecal score of 4
^{††}P-value derived by Fisher's Exact (2-tail) test

The results of this study demonstrate that for both the active BID and active QID treatment groups, a higher percentage of foals treated with the SB-300 active responded to the treatment and had resolution of their diarrhea (fecal scores of 1 or 2) compared with placebo treated animals. More specifically, for the fecal score responder analysis, 68.4% of foals treated with active BID in the 0-72 hour study time period responded (had a score of 1 or 2) compared with 35.3% of placebo-treated animals; and 78.9% of foals treated with active BID in the 0-96 hour study time period responded (had a score of 1 or 2) compared with 47.1% of placebo-treated animals. Similarly, 93.8% of foals treated with active QID in the 0-120 hour study time period responded (had a score of 1 or 2) compared with 46.2% of placebo-treated foals.

In the 0-72 hour study time period, 73.7% of foals treated with active BID had resolution of diarrhea (responder score of 1 or 2) compared with 41.2% of foals treated with placebo. In the 0-96 hour study time period, 84.2% of foals treated with active BID had resolution of diarrhea compared with 52.9% of foals treated with placebo. For foals treated with active QID in the 0-120 hour study time period, 93.8% had resolution of diarrhea compared with 61.5% of foals treated with placebo. The study results demonstrate a high level of efficacy in the resolution of diarrhea in foals treated with the active study agent SB-300.

### Example 6

### Benefit of Croton Lechleri Proanthocyanidin Polymer Extract Composition, Neonorm™, in the Optimization of the Intestinal Microbiome Profile in Pre-Weaned Dairy Calves

This Example describes a study that was conducted to characterize the fecal microbiota (microbiome) of newborn calves experiencing diarrhea induced by enterotoxigenic Escherichia coli (E. coli) and to identify possible relationships between treatment with a standardized, enteric-coated botanical extract derived from the Croton lechleri tree, i.e., the product Neonorm™ Calf, and an altering of the intestinal microbiota profiles of the calves. The microbiome is a community of microorganisms, also termed microbiota, that live normally in the gut and are vital to maintenance of gut health. Neonorm™ Calf specifically addresses the normalization of fecal formation and ion and water flow in the intestinal lumen of newborn dairy calves.

The objective of the microbiome study was to characterize the fecal microbiota of newborn calves experiencing diarrhea induced by enterotoxigenic Escherichia coli (E. coli) and identify possible relationships of treatment with a standardized, enteric-coated botanical extract derived from the Croton lechleri tree, the key composition of Neonorm™ Calf, and the altering of the intestinal microbiota profiles of the calves. As described in hereinabove, Neonorm™ Calf is formulated and has been clinically tested to address the normalization of fecal formation and ion and water flow in the intestinal lumen of newborn dairy calves.

The microbiome study was designed to analyze a subset of randomly selected fecal samples collected during a 2013 challenge study conducted by the College of Veterinary Medicine at Cornell University, titled “Effect of Crofelemer Extract on Severity.” The results of the 2013 study suggested that Neonorm™ Calf significantly increased the fecal dry matter of neonatal calves with experimentally-induced enterotoxigenic E. coli diarrhea, and indicated a benefit of Neonorm™ Calf in supporting weight gain in calves. Similar findings are also described in Examples 3 and 4 above. These studies support the benefits of Neonorm™ in reducing water loss associated with diarrhea and supporting weight gain in preweaned dairy calves.

As described hereinabove, the C. lechleri derived proanthocyanidin extract product, Neonorm™, such as Neonorm™ Calf, is an enteric-coated tablet designed to be orally administered to preweaned dairy calves twice daily for three days. The product acts locally in the gut; is minimally absorbed systemically; and does not alter gastrointestinal motility. To date, the product has shown no significant effects on normally functioning intestinal ion channels and electrolyte or fluid transport, nor shown any side effects different from placebo. As a result, fecal formation is normalized in a short period of time, weight loss is mitigated, and supportive care costs and rehydration therapies such as oral rehydration solution (ORS) are reduced.

For the microbiome study, a total of 28 calves were randomly selected from Holstein newborn bull calves of the 2013 study, as follows: 15 Placebo (CTR) and 13 Enteric-coated SB-300 (ECROF). Fecal samples were collected twice daily as follows: pre-challenge (day 1), during treatment (days 2 to 7), and after treatment cessation (days 8 to 24). The study utilized Next Generation Sequencing to characterize the fecal microbiota of the diarrheic dairy bull calves that were experimentally challenged with enterotoxigenic Escherichia coli to determine whether SB-300 treatment altered or otherwise affected the intestinal microbiota profiles of treated animals. Next generation sequencing, or high throughput sequencing, is known and practiced in the art, for example, as described in A. Grada and K. Weinbrecht, 2013, Journal of Investigative Dermatology, 133:1-4.

More specifically, analysis of the microbiomes showed that the most prevalent phyla, regardless of the treatment group, were Firmicutes, Proteobacteria, Bacteroidetes, Fusobacteria, and Actinobacteria. The baseline samples collected were surprisingly abundant in bacterial DNA. For every calf enrolled in this study, at first sample collection, calves were fed deprived. Proteobacteria were found to be more prevalent in calves enrolled in the enteric-Coated SB-300 group (ECROF). However, throughout the study period, no significant differences of this phylum was detected between treatment groups. Actinobacteria and Tenericutes were found to be more abundant in enteric-coated SB-300 treated calves at the third day of life (7ₜₕ sample) and the second day of life (4th sample), respectively.

The 30 most prevalent bacterial genera in fecal samples collected throughout the study period were also determined. Baseline samples had slightly different relative abundances between calves enrolled in the control and enteric-coated SB-300 groups. For that reason, the measurement of relative abundance change from the baseline abundance was used to enlighten the mechanism in which treatment could be affecting the change in the intestinal microbial profile.

Calves in the control group presented a baseline sample with high prevalence of Streptococcus, Lactobacillus, Bacteroides, Akkermansia, Blautia, and Enterococcus respectively. However, calves enrolled in the enteric-coated SB-300 group had a slightly different microbiome profile; the microbiota was dominated by Streptococcus, Bacteroides, Serratia, Escherichia, Enterococcus and Lactobacillus respectively.

Interestingly, some bacterial genera had a different evolution in their relative abundance following the course of the study. Calves treated with enteric-coated SB-300 presented a relative increase in Faecalibacterium, a bacteria genus that is beneficial to the host, when compared to control calves. In addition, calves treated with enteric-coated SB-300 had a higher relative abundance of the Faecalibacterium bacteria genus after treatment cessation. Bifidobacterium, which is also a genus that comprises a vast species of probiotic bacteria, had a higher relative abundance increase from baseline levels for enteric-coated SB-300 treated calves when compared to control calves.

In this study, all newborn Holstein bull calves were challenged with an inoculum of enterotoxigenic Escherichia coli. Accordingly, the control animals were challenged with E. coli, but did not receive enteric-coated SB-300. Thus, the E. coli challenge could have altered the natural course of the microbiome evolution in milk-fed calves. It could also be possible that the challenge differently transformed the initial bacterial community and that the later interactions between some bacterial genera, were changed in their representation as part of the intestinal microbiome.

The results of the microbiome study demonstrated that the relative abundance of Faecalibacterium, a bacterial genus regarded as beneficial to the host, increased in Neonorm™-treated calves when compared to control calves that had not been treated with Neonorm™. In addition, Neonorm™ treated calves had a higher relative abundance of Faecalibacterium following cessation of treatment. The results further support that the beneficial prebiotic mechanism and/or property of Neonorm™ may supplement and is potentially synergistic with the anti-secretory and weight gain benefits of the product.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method of treating a neonatal or young non-human animal having diarrhea associated with enteropathogenic infection, the method comprising orally administering to the neonatal or young non-human animal in need thereof a pharmaceutical composition comprising an aqueous soluble proanthocyanidin polymer from Croton lechleri, wherein the composition is formulated as a bolus or as a reconstituted powder and administered to the neonatal or young non-human animal in an amount of at least 40 mg to 300 mg per day for two or more consecutive days.

2. The method according to claim 1, wherein the neonatal or young non-human animal is selected from a bovine calf, a camel calf, a buffalo calf, a bison calf, a lamb, a kid, a foal, or a piglet.

3. The method according to claim 2, wherein the neonatal or young non-human animal is a bovine calf.

4. The method according to claim 2, wherein the neonatal or young non-human animal is a camel calf.

5. The method according to any one of claims 1 to 4, wherein the non-human animal is unweaned.

6. The method according to any one of claims 1 to 5, wherein the proanthocyanidin polymer composition is administered twice daily for three consecutive days.

7. The method according to any one of claims 1 to 6, wherein the enteropathogenic infection is with one or more of E. coli, rotavirus, or coronavirus.

8. The method according to claim 7, wherein the enteropathogenic infection is additionally with Salmonella spp. and/or Cryptosporidia.

9. The method according to any one of claims 1 to 8, wherein the proanthocyanidin polymer composition is administered as a powder reconstituted with oral electrolytes, milk or a milk substitute, physiological saline, or water.

10. The method according to any one of claims 1 to 9, wherein the proanthocyanidin polymer composition is administered as a bolus.

11. The method according to any one of claims 1 to 8, wherein the proanthocyanidin polymer composition is administered in animal feed.

12. The method according to any one of claims 1 to 11, wherein the non-human animal is less than two weeks of age.

13. The method according to any one of claims 1 to 11, wherein the non-human animal is two to four weeks of age.

14. The method according to any one of claims 1 to 13, wherein the composition is administered to the neonatal or young non-human animal in an amount of at least 30 mg to 350 mg.

15. The method according to any one of claims 1 to 14, wherein the composition is administered to the neonatal or young non-human animal in an amount of 250 mg.

16. The method according to any one of claims 1 to 13, wherein the neonatal or young non-human animal is a lamb or a kid, and the composition is administered in an amount of 40 mg or 50 mg.

17. The method according to any one of claims 1 to 16, wherein the neonatal or young non-human animal is approximately 30 to 50 kg in weight.

18. The method according to claim 3, wherein the bovine calf is approximately 30 to 40 kg in weight.

19. The method according to claim 4, wherein the camel calf is approximately 40 to 50 kg in weight.

20. The method according to any one of claims 1 to 19, wherein the proanthocyanidin polymer is administered as an enteric coated pharmaceutical composition.

21. The method according to any one of claims 1 to 19, wherein the proanthocyanidin polymer is administered as a non-enteric coated pharmaceutical composition.

22. The method according to any one of claims 1 to 21, wherein the proanthocyanidin polymer is selected from the group consisting of SB-300, SP 303, and crofelemer.

23. A method of improving weight gain or reducing mortality in neonatal non-human animal, said method comprising administering to said neonatal non-human animal a pharmaceutical composition comprising an aqueous soluble proanthocyanidin polymer from Croton lechleri, wherein the composition is formulated as a bolus or as a reconstituted powder and administered to the neonatal or young non-human animal in an amount effective to improve weight gain or reduce mortality for at least one day between the first and fourth days after birth of said neonatal non-human animal.

24. The method according to claim 23, wherein the neonatal non-human animal is selected from a bovine calf, a camel calf, a buffalo calf, a bison calf, a lamb, a kid, a foal, or a piglet.

25. The method according to claim 24, wherein the neonatal non-human animal is a bovine calf.

26. The method according to claim 24, wherein the neonatal non-human animal is a camel calf.

27. The method according to any one of claims 23 to 26, wherein the proanthocyanidin polymer composition is administered for at least three consecutive days.

28. The method according to any one of claims 23 to 27, wherein the proanthocyanidin polymer composition is administered as a powder reconstituted with oral electrolytes, milk or a milk substitute, physiological saline, or water.

29. The method according to any one of claims 23 to 28, wherein the composition is administered to the neonatal non-human animal in an amount of between 30 mg to 350 mg.

30. The method according to any one of claims 23 to 28, wherein the composition is administered to the neonatal animal in an amount of 250 mg.

31. The method according to any one of claims 23 to 30, wherein the proanthocyanidin polymer is administered as an enteric coated pharmaceutical composition.

32. The method according to any one of claims 23 to 30, wherein the proanthocyanidin polymer is administered as a non-enteric coated pharmaceutical composition.

33. The method according to any one of claims 23 to 32, wherein the proanthocyanidin polymer is selected from the group consisting of SB-300, SP 303, and crofelemer.

34. A method of treating a neonatal or unweaned equine animal for diarrhea associated with enteropathogenic infection, the method comprising orally administering to the animal a pharmaceutical composition comprising an aqueous soluble proanthocyanidin polymer from Croton lechleri, wherein the composition is provided in a form selected from a bolus, a reconstituted powder, or a gel, and is administered to the animal in an amount of at least 100 mg for two or more consecutive days.

35. The method according to claim 34, wherein the animal is infected with bacteria, viruses and protozoa, which infection induced the diarrhea.

36. The method according to claim 34 or 35, wherein the proanthocyanidin polymer composition is administered to the animal in an amount of at least 250 mg.

37. The method according to any one of claims 34 to 36, wherein the proanthocyanidin polymer composition is in the form of a gel contained in a delivery device.

38. The method according to claim 37, wherein the delivery device is a syringe.

39. The method according to claim 37 or claim 38, wherein the gel comprises polymeric microparticles or nanoparticles containing the composition.

40. The method according to claim 39, wherein the polymeric microparticles or nanoparticles are pH-sensitive.

41. The method according to any one of claims 34 to 40, wherein the animal is less than two weeks of age.

42. The method according to any one of claims 34 to 41, wherein the animal is approximately 30 to 50 kg in weight.

43. The method according to any one of claims 34 to 42, wherein the proanthocyanidin polymer is administered as an enteric coated pharmaceutical composition.

44. The method according to any one of claims 34 to 42, wherein the proanthocyanidin polymer is administered as a non-enteric coated pharmaceutical composition.

45. The method according to any one of claims 34 to 44, wherein the proanthocyanidin polymer is selected from the group consisting of SB-300, SP 303, and crofelemer.

46. A method of treating or preventing diarrhea in a neonatal or young equine foal, the method comprising orally administering to the foal in need thereof a pharmaceutical composition comprising an aqueous soluble proanthocyanidin polymer from Croton lechleri, wherein the composition is formulated as a paste and is orally administered to the foal in an amount of 2 mg/kg per day two times per day for two or more consecutive days, or three times per day for two or more consecutive days, or four times per day for two or more consecutive days.

47. The method according to claim 46, wherein the composition is formulated as a paste and is orally administered to the foal in an amount of 2 mg/kg per day two times per day, or three times per day, or four times per day for three consecutive days.

48. The method according to claim 46 or claim 47, wherein the paste is administered two times a day, twelve hours apart, for three consecutive days, or four times a day for three consecutive days.

49. The method according to any one of claims 46 to 48, wherein the proanthocyanidin polymer is selected from the group consisting of SB-300, SP 303, and crofelemer.

50. The method according to any one of claims 46 to 49, wherein the paste comprises beads comprising enterically coated SB-300.

51. The method according to any one of claims 46 to 50, wherein the paste is contained in a delivery device.

52. The method according to claim 51, wherein the delivery device is a syringe.

53. The method according to any one of claims 46 to 52, wherein the oral administration comprises applying the paste to the roof of the foal's mouth.

54. A method of inducing an intestinal microbiota favoring normal stool formation, fecal consistency and diarrhea reduction or prevention in a neonatal, non-human animal suffering from diarrhea and its accompanying symptoms, said method comprising orally administering to the neonatal, non-human animal a pharmaceutical composition comprising an enterically coated, aqueous soluble proanthocyanidin polymer from Croton lechleri or a Croton lechleri-derived botanical extract at least once a day prior to a meal, for at least two days.

55. A method of treating a bovine calf suffering from, or at risk of developing, diarrhea and dehydration, and maintaining normal fecal consistency and preventing dehydration in the calf following cessation of treatment, said method comprising orally administering to a preweaned calf a pharmaceutical composition comprising an enterically coated, aqueous soluble proanthocyanidin polymer from Croton lechleri or a Croton lechleri-derived botanical extract two times per day prior to a meal, for two to three days.

56. The method according to claim 54, wherein the neonatal non-human animal is a bovine calf.

57. The method according to claim 56, wherein the neonatal non-human animal is preweaned.

58. The method according to any one of claim 54, 56, or 57, wherein the neonatal, non-human animal is orally administered the pharmaceutical composition comprising an enterically coated, aqueous soluble proanthocyanidin polymer from Croton lechleri or a Croton lechleri-derived botanical extract two times per day, or three times per day, or four times per day.

59. The method according to any one of claims 54 to 58, wherein the neonatal non-human animal or the calf is administered an enterically coated, Croton lechleri-derived botanical extract.

60. The method according to any one of claims 54 to 59, wherein the neonatal non-human animal or the calf is administered an enterically coated, Croton lechleri-derived botanical extract two times per day for three days, or three times per day for three days, or four times per day for three days.

61. The method according to any one of claims 54 to 60, wherein dry fecal consistency, lack of dehydration and/or a healthy gastrointestinal microbiota of the neonatal non-human animal or the calf is maintained and sustained for at least two to three weeks following cessation of treatment with the pharmaceutical composition comprising an enterically coated, aqueous soluble proanthocyanidin polymer from Croton lechleri or the Croton lechleri-derived botanical extract.

62. The method according to any one of claims 54 to 61, wherein the enterically coated, aqueous soluble proanthocyanidin polymer from Croton lechleri or the Croton lechleri-derived botanical extract is administered to the neonatal non-human animal or the calf as a bolus.

63. The method according to any one of claims 54 to 62, wherein the enterically coated, aqueous soluble proanthocyanidin polymer from Croton lechleri or the Croton lechleri-derived botanical extract is administered to the neonatal non-human animal or the calf at the first sign or indication of diarrhea.

64. A method of promoting or maintaining a beneficial intestinal microbiota in a non-human animal, said method comprising administering to said non-human animal a prebiotic composition comprising a botanical extract derived from a Croton or Calophyllum species containing polyphenols in an amount effective to promote or maintain a beneficial intestinal microbiota.

65. The method according to claim 64 in which the prebiotic composition comprises a botanical extract derived from Croton lechleri containing polyphenols

66. The method according to claim 65 in which the prebiotic composition comprises SB-300 or SP-303.

67. The method according to any of claims claims 64 to 66, wherein the non-human animal is selected from a bovine, a camel, a buffalo, a bison, a sheep, a goat, a horse or a pig.

68. The method according to any of claims 64 to 66 wherein the non-human animal is selected from a fowl, a dog, a cat, a rodent or an exotic mammal.

69. The method according to any one of claims 64 to 68 wherein the non-human animal is a neonate.

70. The method according to any one of claims 64 to 68 wherein the non-human animal is a juvenile.

71. The method according to any one of claims 64 to 68 wherein the non-human animal is an adult.

72. The method according to any one of claims 64 to 71 resulting in improved intestinal health, increased weight gain, reduced morbidity, reduced mortality, or improved stool formation.

73. The method according to any one of claims 64 to 72, wherein the prebiotic composition is administered for at least three consecutive days.

74. The method according to any one of claims 64 to 73 wherein said prebiotic composition is administered chronically to maintain intestinal health.

75. The method according to any one of claims 64 to 74, wherein the prebiotic composition is formulated as animal feed.

76. The method according to any one of claims 64 to 74, wherein the composition is formulated in a medicinal feed block.

77. The method according to any one of claims 64 to 76, wherein the botanical extract is administered as an enteric coated prebiotic composition.

78. The method according to any one of claims 64 to 76, wherein the botanical extract is administered as a non-enteric coated prebiotic composition.

79. The method according to claim 77 or 78, wherein the administration results in increases in the levels of Bifidobacterium and/or Faecalibacterium in the gut biome.

80. The method according to any one of claims 64 to 79 further comprising administering a probiotic to said non-human animal.

81. The method according to any one of claims 64 to 80, wherein administration of said prebiotic composition is associated with an increase in probiotic bacterial genera in the non-human animal's microbiome.

82. The method according to claim 81, wherein the probiotic bacteria genera include Bifidobacterium and Faecalibacterium.
